(19) United States
(12) Patent Application Publication
(10) Pub. No.: US 2026/0103758 A1
(43) Pub. Date: Apr. 16, 2026

(54) METHODS AND BIOMARKERS FOR TREATING BREAST CANCER

(71) Applicant: KING ABDULAZIZ UNIVERSITY, Jeddah (SA)

(72) Inventors: Sajjad KARIM, Jeddah (SA); Haneen Reda BANJAR, Jeddah (SA); Md Shahid ANSARI, Jeddah (SA); Adeel CHAUDHARY, Jeddah (SA); Md Shahid IQBAL, Jeddah (SA); Zeenat MIRZA, Jeddah (SA)

(73) Assignee: KING ABDULAZIZ UNIVERSITY, Jeddah (SA)

(21) Appl. No.: 18/916,490

(22) Filed: Oct. 15, 2024

(51) Int. Cl. C12Q 1/6886

(52) U.S. Cl. CPC C12Q 1/6886; C12Q 2600/106; C12Q 2600/112; C12Q 2600/158

(57) ABSTRACT

A method for treating a breast cancer includes assaying a breast tumor sample from the subject for gene expression levels of a diagnostic gene set including at least two genes selected from the group consisting of CDCA7, HORMAD1, AR, MLPH, TFF3, AGR2, ATL2, ACOX2, REEP6, CAPN8, FOXA1, CT83, ART3, PPP1R14C, and PRR15, and a prognostic gene set including at least two genes selected from the group consisting of CDCA7, ATL2, REEP6, CAMK2N1, REEP1, GABBR1, IL12RB2, ARNTL2, AZGP1, and FBP1. The method includes selecting a treatment option which is least one selected from the group including surgery and an anticancer agent based on the gene expression levels. The method further includes treating the patient with the treatment option.

### STATEMENT OF ACKNOWLEDGEMENT

This research work was funded by Institutional Fund Projects under grant no (IFPRC-125-141-2020). Therefore, authors gratefully acknowledge technical and financial support from the Ministry of Education and King Abdulaziz University, Jeddah, Saudi Arabia.

### BACKGROUND

### Technical Field

The present invention relates to a method and biomarkers for treating breast cancer, and particularly to biomarkers for triple-negative breast cancer (TNBC) subtypes, particularly basal-like breast cancer (BLBC) subtype.

### Description of Related Art

The ‘background’ description provided herein presents the context of the disclosure generally. The work of the presently named inventors, to the extent it is described in this background section, as well as aspects of the description that may not otherwise qualify as prior art at the time of filing, are neither expressly nor impliedly admitted as prior art against the present invention.

Breast cancer (BC) represents the most common cancer diagnosed in women, with approximately 2.3 million new cases and 685,000 deaths recorded globally in 2020. Current projections suggest a notable increase in BC incidence by 2040, highlighting the urgent need for effective treatment strategies. The complexity of BC arises from its heterogeneous nature, characterized by several distinct molecular subtypes, each exhibiting unique clinical and prognostic characteristics. This variability underscores the critical importance of molecular classification in developing personalized treatment approaches.

Traditionally, the management of BC has relied heavily on histopathological evaluations and immunohistochemical markers. This has led to the classification of BC into several subtypes, including luminal A, luminal B, HER2-enriched, triple-negative, and normal-like. While these classifications provide useful prognostic insights, many have not been thoroughly validated against gene expression profiles. This lack of validation poses a risk of misidentification and therefore ineffective or counterproductive treatment, particularly concerning triple-negative breast cancer (TNBC), which is notably resistant to standard targeted therapies.

Recent advancements in transcriptomic analysis have paved the way for creating various gene signatures to improve subtype classification and enhance predictions regarding treatment responses. However, despite the introduction of tools such as OncotypeDX, MammaPrint, and PAM50, significant barriers remain to translating these findings into clinical practice and tangible improvements in treatments and patient outcomes. Key challenges include the need for comprehensive validation and the requirement for larger sample sizes.

Accordingly, one object of the present disclosure is to address the pressing need for the identification of biomarkers that can enhance treatment strategies specifically for breast cancer subtypes, particularly BLBC, and the use of such biomarkers in cancer treatment.

### SUMMARY

According to a first aspect, the present disclosure is related to a method for treating breast cancer in a subject in need thereof. In some embodiments, the method includes assaying a breast tumor sample from the subject for gene expression levels of a diagnostic gene set including at least two genes selected from the group consisting of CDCA7, HORMAD1, AR, MLPH, TFF3, AGR2, ATL2, ACOX2, REEP6, CAPN8, FOXA1, CT83, ART3, PPP1R14C, and PRR15, and a prognostic gene set including at least two genes selected from the group consisting of CDCA7, ATL2, REEP6, CAMK2N1, REEP1, GABBR1, IL12RB2, ARNTL2, AZGP1, and FBP1. In some embodiments, the method includes selecting a treatment option, which is at least one selected from the group, including surgery and an anticancer agent based on the gene expression levels. In some embodiments, the method further includes treating the subject with the treatment option.

In some embodiments, the breast cancer is determined to be a basal-like breast cancer based on the gene expression levels of the diagnostic gene set.

In some embodiments, the anticancer agent is at least one selected from the group consisting of an anticancer agent targeting MSLN, an anticancer agent targeting TFF1, an anticancer agent targeting KRT16, an anticancer agent targeting IMPA2, an anticancer agent targeting FRPI, an anticancer agent targeting AR.

In some embodiments, the anticancer agent is selected based on the gene expression levels of the prognostic gene set.

In some embodiments, the anticancer agent targeting MSLN is amatuximab.

In some embodiments, the anticancer agent targeting TFF1 is at least one selected from the group consisting of raloxifene and afimoxifene.

In some embodiments, the anticancer agent targeting KRT16 is at least one selected from the group consisting of zinc, zinc acetate, and zinc chloride.

In some embodiments, the anticancer agent targeting IMPA2 is at least one selected from the group consisting of lithium cation, lithium citrate, lithium succinate, and lithium carbonate.

In some embodiments, the anticancer agent targeting FBP1 is at least one selected from the group consisting of adenosine phosphate, Mdl-29951, fructose-6-phosphate, MB-07803, and managlinat dialanetil.

In some embodiments, the anticancer agent targeting AR is at least one selected from the group consisting of diethylstilbestrol, levonorgestrel, progesterone, spironolactone, flutamide, oxandrolone, and fluphenazine.

In some embodiments, the diagnostic gene set includes at least five genes selected from the group consisting of CDCA7, HORMAD1, AR, MLPH, TFF3, AGR2, ATL2, ACOX2, REEP6, CAPN8, FOXA1, CT83, ART3, PPP1R14C, and PRR15.

In some embodiments, the diagnostic gene set includes at least seven genes selected from the group consisting of CDCA7, HORMAD1, AR, MLPH, TFF3, AGR2, ATL2, ACOX2, REEP6, CAPN8, FOXA1, CT83, ART3, PPP1R14C, and PRR15.

In some embodiments, the diagnostic gene set includes at least nine genes selected from the group consisting of CDCA7, HORMAD1, AR, MLPH, TFF3, AGR2, ATL2, ACOX2, REEP6, CAPN8, FOXA1, CT83, ART3, PPP1R14C, and PRR15.

In some embodiments, the diagnostic gene set includes at least eleven genes selected from the group consisting of CDCA7, HORMAD1, AR, MLPH, TFF3, AGR2, ATL2, ACOX2, REEP6, CAPN8, FOXA1, CT83, ART3, PPP1R14C, and PRR15.

In some embodiments, the diagnostic gene set includes CDCA7, HORMAD1, AR, MLPH, TFF3, AGR2, ATL2, ACOX2, REEP6, CAPN8, FOXA1, CT83, ART3, PPP1R14C, and PRR15.

In some embodiments, the prognostic gene set includes at least three genes selected from the group consisting of CDCA7, ATL2, REEP6, CAMK2N1, REEP1, GABBR1, IL12RB2, ARNTL2, AZGP1, and FBP1.

In some embodiments, the prognostic gene set includes at least five genes selected from the group consisting of CDCA7, ATL2, REEP6, CAMK2N1, REEP1, GABBR1, IL12RB2, ARNTL2, AZGP1, and FBP1.

In some embodiments, the prognostic gene set includes at least seven genes selected from the group consisting of CDCA7, ATL2, REEP6, CAMK2N1, REEP1, GABBR1, IL12RB2, ARNTL2, AZGP1, and FBP1.

In some embodiments, the prognostic gene set includes at least nine genes selected from the group consisting of CDCA7, ATL2, REEP6, CAMK2N1, REEP1, GABBR1, IL12RB2, ARNTL2, AZGP1, and FBP1.

In some embodiments, the prognostic gene set includes CDCA7, ATL2, REEP6, CAMK2N1, REEP1, GABBR1, IL12RB2, ARNTL2, AZGP1, and FBP1.

The foregoing general description of the illustrative embodiments and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 is a flow chart depicting a method of treating a breast cancer in a subject, according to some embodiments.

FIG. 2A is a volcano plot depicting basal-like vs. HER2, showing GABRP, HORMAD1, OVOS2, and FOXC1 as upregulated genes and ERBB2, MEN1, PGAP3, FOXA1, and MLPH as downregulated genes, according to some embodiments.

FIG. 2B is a volcano plot depicting basal-like vs. LumB, showing BCL11A, GABRP, HORMAD1, and FOXC1 as upregulated genes and AGR3, AGR2, FOXA1, MLPH, and CA12 as downregulated genes, according to some embodiments.

FIG. 2C is a volcano plot depicting basal-like vs. LumA showing CDC20, TTK, CENPA, CDC7, and FOXC1 as upregulated and AGR3, AR, MLPH and CA12 as downregulated genes, according to some embodiments.

FIG. 2D is a volcano plot depicting basal-like vs. normal-like breast cancer, showing TTK, MELK, CENPA, CDC7, and FOXC1 as upregulated genes and AGR3, AR, MLPH, and CA12 as downregulated genes, according to some embodiments.

FIG. 2E is a volcano plot depicting basal-like vs. normal breast tissue, showing NUF2, KIF14, TPX2, and ANLN as upregulated genes and AR, MLPH, AGR2, and FOXA1 as downregulated genes, according to some embodiments.

FIG. 2F is a volcano plot depicting basal-like vs. combined non-basal-like BC showing EN1, CT83, TTK, and CDC7 as upregulated and AGR3, AR, MLPH and CA12 as downregulated, according to some embodiments.

FIG. 3A is a Venn diagram showing 94 differentially expressed genes for basal-like subtype vs. HER2, LumA, LumB, normal-like, and normal breast cancer, according to some embodiments.

FIG. 3B is a Venn diagram showing 61 differentially expressed genes exclusive for basal-like subtypes, according to some embodiments.

FIG. 4A is a principal component analysis (PCA) plot based on differentially expressed genes, according to some embodiments.

FIG. 4B is a PCA plot based on 61 basal-like filtered genes, according to some embodiments.

FIG. 5A depicts HeatMap of 337 DEGs of breast cancer, according to some embodiments.

FIG. 5B depicts HeatMap of 61 DEGs of basal-like, according to some embodiments.

FIG. 5C depicts HeatMap of diagnostic gene signature of basal-like subtype, according to some embodiments.

FIG. 6 depicts average expression values of diagnostic gene signatures for basal-like subtypes, including upregulated genes (HORMAD1, CDCA7, ATL2, ART3, CT83, PPP1R14C), downregulated genes (ACOX2, AR, AGR2, CAPN8, FOXA1, MLPH, PRR15, REEP6, TFF3), and cancer genes (SCARA5) down expressed in breast cancer subtypes, according to some embodiments.

FIG. 7A is a Kaplan-Meier survival graph of CDCA7 prognostic gene-signature depicting significance in overall survival using StGallen basal-like subtype of breast cancer, according to some embodiments.

FIG. 7B is a Kaplan-Meier survival graph of ATL2 prognostic gene-signature depicting significance in overall survival using StGallen basal-like subtype of breast cancer, according to some embodiments.

FIG. 7C is a Kaplan-Meier survival graph of REEP6 prognostic gene-signature depicting significance in overall survival using StGallen basal-like subtype of breast cancer, according to some embodiments.

FIG. 7D is a Kaplan-Meier survival graph of CAMK2N1 prognostic gene-signature depicting significance in overall survival using StGallen basal-like subtype of breast cancer, according to some embodiments.

FIG. 7E is a Kaplan-Meier survival graph of REEP1 prognostic gene-signature depicting significance in overall survival using StGallen basal-like subtype of breast cancer, according to some embodiments.

FIG. 7F is a Kaplan-Meier survival graph of GABBR1 prognostic gene-signature depicting significance in overall survival using StGallen basal-like subtype of breast cancer, according to some embodiments.

FIG. 7G is a Kaplan-Meier survival graph of IL12RB2 prognostic gene-signature depicting significance in overall survival using StGallen basal-like subtype of breast cancer, according to some embodiments.

FIG. 7H is a Kaplan-Meier survival graph of ARNTL2 prognostic gene-signature depicting significance in overall survival using StGallen basal-like subtype of breast cancer, according to some embodiments.

FIG. 7I is a Kaplan-Meier survival graph of AZGP1 prognostic gene-signature depicting significance in overall survival using StGallen basal-like subtype of breast cancer, according to some embodiments.

FIG. 7J is a Kaplan-Meier survival graph of FUBP1 prognostic gene-signature depicting significance in overall survival using StGallen basal-like subtype of breast cancer, according to some embodiments.

FIG. 7K is a Kaplan-Meier survival graph of CDCA7 prognostic gene-signature depicting significance in relapse-free survival using StGallen basal-like subtype of breast cancer, according to some embodiments.

FIG. 7L is a Kaplan-Meier survival graph of ATL2 prognostic gene-signature depicting significance in relapse-free survival using StGallen basal-like subtype of breast cancer, according to some embodiments.

FIG. 7M is a Kaplan-Meier survival graph of REEP6 prognostic gene-signature depicting significance in relapse-free survival using StGallen basal-like subtype of breast cancer, according to some embodiments.

FIG. 7N is a Kaplan-Meier survival graph of CAMK2N1 prognostic gene-signature depicting significance in relapse-free survival using StGallen basal-like subtype of breast cancer, according to some embodiments.

FIG. 7O is a Kaplan-Meier survival graph of REEP1 prognostic gene-signature depicting significance in relapse-free survival using StGallen basal-like subtype of breast cancer, according to some embodiments.

FIG. 7P is a Kaplan-Meier survival graph of GABBR1 prognostic gene-signature depicting significance in relapse-free survival using StGallen basal-like subtype of breast cancer, according to some embodiments.

FIG. 7Q is a Kaplan-Meier survival graph of IL12RB2 prognostic gene-signature depicting significance in relapse-free survival using StGallen basal-like subtype of breast cancer, according to some embodiments.

FIG. 7R is a Kaplan-Meier survival graph of ARNTL2 prognostic gene-signature depicting significance in relapse-free survival using StGallen basal-like subtype of breast cancer, according to some embodiments.

FIG. 7S is a Kaplan-Meier survival graph of AZGP1 prognostic gene-signature depicting significance in relapse-free survival using StGallen basal-like subtype of breast cancer, according to some embodiments.

FIG. 7T is a Kaplan-Meier survival graph of FBP1 prognostic gene-signature depicting significance in relapse-free survival using StGallen basal-like subtype of breast cancer, according to some embodiments.

FIGS. 8A-8H depicts area under the receiver operating characteristic (AUC-ROC) graphs of external validation showing the reliability of the developed machine learning model of basal-like diagnostic gene-signature, according to some embodiments.

FIG. 9A depicts the over-expression of ART3 genes in basal-like breast cancer at the GEO database, according to some embodiments.

FIG. 9B depicts over-expression of ATL2 genes in basal-like breast cancer at GEO database, according to some embodiments.

FIG. 9C depicts the over-expression of PPP1R14C genes in basal-like breast cancer in the GEO database, according to some embodiments.

FIG. 9D depicts over-expression of ATL2 genes in basal-like breast cancer at TCGA database, according to some embodiments.

FIG. 9B depicts over-expression of ART3 genes in basal-like breast cancer at TCGA database, according to some embodiments.

FIG. 9F depicts over-expression of PPP1R14C genes in basal-like breast cancer at TCGA database, according to some embodiments.

FIG. 9G depicts over-expression of ART3 genes in basal-like breast cancer at CPTAC database, according to some embodiments.

FIG. 9H depicts over-expression of ATL2 genes in basal-like breast cancer at CPTAC database, according to some embodiments.

FIG. 9I depicts over-expression of PPP1R14C genes in basal-like breast cancer at CPTAC database, according to some embodiments.

FIG. 9J depicts under-expression of ACOX2 genes in basal-like breast cancer at GEO database, according to some embodiments.

FIG. 9K depicts under-expression of AGR2 genes in basal-like breast cancer at GEO database, according to some embodiments.

FIG. 9L depicts under-expression of AR genes in basal-like breast cancer at GEO database, according to some embodiments.

FIG. 9M depicts under-expression of ACOX2 genes in basal-like breast cancer at TCGA database, according to some embodiments.

FIG. 9N depicts under-expression of AGR2 genes in basal-like breast cancer at TOGA database, according to some embodiments.

FIG. 9O depicts under-expression of AR genes in basal-like breast cancer at TCGA database, according to some embodiments.

FIG. 9P depicts under-expression of ACOX2 genes in basal-like breast cancer at CPTAC database, according to some embodiments.

FIG. 9Q depicts under-expression of AGR2 genes in basal-like breast cancer at CPTAC database, according to some embodiments.

FIG. 9R depicts under-expression of AR genes in basal-like breast cancer at CPTAC database, according to some embodiments.

FIG. 10 depicts real time qPCR of diagnostic and prognostic gene-signatures, according to some embodiments.

### DETAILED DESCRIPTION

In the drawings, reference numerals designate identical or corresponding parts throughout the several views. Further, as used herein, the words ‘a,’ ‘an,’ and the like generally mean ‘one or more’ unless stated otherwise.

Furthermore, the terms ‘approximately,’ ‘approximate,’ ‘about,’ and similar terms generally refer to ranges that include the identified value within a margin of 20%, 10%, or preferably 5%, and any values there between.

The term “treating” or “treatment” as used herein means the treating or treatment of a disease or medical condition in a patient, such as a mammal (particularly a human) that includes: ameliorating the disease or medical condition, such as, eliminating or causing regression of the disease or medical condition in a patient; suppressing the disease or medical condition, for example by, slowing or arresting the development of the disease or medical condition in a patient; or alleviating a symptom of the disease or medical condition in a patient. In some embodiments, the subject is a mammalian subject. In one embodiment, the subject is a human. ‘Treating’ or ‘treatment’ of a disease includes preventing the disease from occurring in a subject that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease). Regarding cancer or hyperplasia, these terms may be associated with an increase in the life expectancy of an individual affected with cancer or reduction of one or more of the symptoms of the disease. In specific embodiments, such terms may refer to one, two or three or more results following the administration of one, two, three or more therapies: (1) a stabilization, reduction or elimination of the cancer stem cell population; (2) a stabilization, reduction or elimination in the cancer cell population; (3) a stabilization or reduction in the growth of a tumor or neoplasm; (4) an impairment in the formation of a tumor; (5) eradication, removal, or control of primary, regional and/or metastatic cancer; (6) a reduction in mortality; (7) an increase in disease-free, relapse-free, progression-free, and/or overall survival, duration, or rate; (8) an increase in the response rate, the durability of response, or number of patients who respond or are in remission; (9) a decrease in hospitalization rate, (10) a decrease in hospitalization lengths, (11) the size of the tumor is maintained and does not increase or increases by less than 10%, preferably less than 5%, preferably less than 4%, preferably less than 2%, and (12) an increase in the number of patients in remission. In certain embodiments, such terms refer to a stabilization or reduction in cancer stem cell population. In some embodiments, such terms refer to a stabilization or reduction in the growth of cancer cells. In some embodiments, such terms refer to stabilization or reduction in cancer stem cell population and a reduction in the cancer cell population. In some embodiments, such terms refer to a stabilization or reduction in the growth and or formation of a tumor. In some embodiments, such terms refer to the eradication, removal, or control of primary, regional, or metastatic cancer (e.g., the minimization or delay of the spread of cancer). In some embodiments, such terms refer to a reduction in mortality and/or an increase in the survival rate of a patient population. In further embodiments, such terms refer to an increase in the response rate, the durability of response, or the number of patients who respond or are in remission. In some embodiments, such terms refer to a decrease in the hospitalization rate of a patient population and/or a decrease in hospitalization length for a patient population. A treatment can provide a therapeutic benefit such as the eradication or amelioration of one or more of the physiological or psychological symptoms associated with the underlying condition, disease, or disorder such that an improvement is observed in the patient, notwithstanding the fact that the patient may still be affected by the condition.

‘Anti-cancer agent’ and ‘anticancer agent’ or ‘anticancer compound’ are used in accordance with their plain and ordinary meaning and refers to a composition (e.g. compound, drug, antagonist, inhibitor, modulator) having antineoplastic properties, the ability to inhibit the growth or proliferation of cells, or a recognized medical use in the treatment of cancer. In some embodiments, an anti-cancer agent is chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XLS18, CI-1040, PD035901, selumetinib/AZD6244, GSK1120212/trametinib, GDC-0973, ARRY-162, ARRY-300, AZD8330, PD0325901, 00126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflorithine, elemene; emitefur, epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin, gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic; platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin, nagrestip; naloxone+pentazocine, napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid, panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin, piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone BI; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine, tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin I1 (including recombinant interleukin II, or rIL.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol™ (i.e. paclitaxel), Taxotere™, compounds including the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B. Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), LS-4359-P (Pharmacia, i.e. LS-4577), LS-4578 (Pharmacia, i.e. LS-477-P), LS-4477 (Pharmacia), LS-4559 (Pharmacia), RPR-112378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e. ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e. LY-355703), AC-7739 (Ajinomoto, i.e. AVE-8063A and CS-39.HCl), AC-7700 (Ajinomoto, i.e. AVE-8062, AVE-8062A, CS-39-L-Ser.HCl, and RPR-258062A), Vitilevuamide, Tubulysin A, Canadensol, Centaureidin (i.e. NSC-106969), T-138067 (Tularik, i.e. T-67, TL-138067 and TI-138067), COBRA-I (Parker Hughes Institute, i.e. DDE-261 and WHI-261), H10 (Kansas State University), H16 (Kansas State University), Oncocidin A1 (i.e. BTO-956 and DIME), DDE-313 (Parker Hughes Institute), Fijianolide B. Laulimalide, SPA-2 (Parker Hughes Institute), SPA-1 (Parker Hughes Institute, i.e. SPIKET-P), 3-IAABU (Cytoskeleton/Mt. Sinai School of Medicine, i.e. MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e. MF-191). TMPN (Arizona State University), Vanadocene acetylacetonate, T-138026 (Talarik), Monsatrol, Inanocine (i.e. NSC-698666), 3-IAABE (Cytoskeleton/Mt. Sinai School of Medicine), A-204197 (Abbott), T-607 (Tufarik, i.e. T-900607), RPR-115781 (Aventis), Eleutherobins (such as Desmethyleleutherobin, Dessetyleleutherobin, Isoeleutherobin A, and Z-Eleutherobin), Caribacoside, Caribacolin, Halichondrin B, D-64131 (Asta Medica), D-68144 (Asta Medica). Diazonamide A, A-293620 (Abbott), NPI-2350 (Nereus), Taccalonolide A, TUB-245 (Aventis), A-259754 (Abbott), Diozostatin, (−)-Phenylahistin (i.e. NSCL-96F037), D-68838 (Asta Medica), D-68836 (Asta Medica), Myoseverin B, D-43411 (Zentaris, i.e. D-81862), A-289099 (Abbott), A-318315 (Abbott), HTI-286 (i.e. SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g. hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), immunotherapy (e.g., cellular immunotherapy, antibody therapy, cytokine therapy, combination immunotherapy, etc.), radioimmunotherapy (e.g., anti-CD20 monoclonal antibody conjugated to ¹¹¹In, ⁹⁰Y, or ¹³¹I, etc.), immune checkpoint inhibitors (e.g., CTLA4 blockade, PD-1 inhibitors, PD-L1 inhibitors, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™), panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like.

A ‘prognostic gene set’ refers to a defined collection of genes whose expression levels are statistically correlated with clinical outcomes in patients diagnosed with a specific disease, such as breast cancer, and/or on which is utilized to evaluate the likelihood of disease progression, recurrence, or survival, thus serving as a biomarker for prognosis. The expression profiles of genes in a prognostic gene set can inform treatment decisions and guide therapeutic interventions, facilitating personalized medicine by stratifying patients based on their predicted disease course. A prognostic gene set may include both upregulated and downregulated genes. A prognostic gene set may be employed alongside established clinical parameters to enhance the accuracy of prognostic predictions. An expression level of a gene in a prognostic gene set may be quantitatively assessed using methods such as quantitative PCR (qPCR), RNA sequencing, or other molecular biology techniques on biological samples, including tumor tissues. Each gene within the prognostic gene set described herein has been identified through comprehensive statistical analyses of gene expression data that correlate with patient outcomes, demonstrating significant associations with relapse-free survival (RFS) or overall survival (OS).

A ‘diagnostic gene set’ refers to a specified collection of genes whose expression levels are utilized to determine the presence or classification of a disease, such as breast cancer, in a subject. The diagnostic gene set can include biomarkers that enable the differentiation of disease subtypes, facilitating accurate diagnosis and characterization of the disease state (e.g., cancer type or cancer sub-type). The expression profiles of genes in a diagnostic gene set can guide clinical decision-making by providing critical information regarding the underlying biological mechanisms of the disease. A diagnostic gene set may include genes that are differentially expressed in the context of the disease. An expression level of a gene in a diagnostic gene set may be evaluated using molecular techniques such as quantitative PCR (qPCR), RNA sequencing, or other genomic methodologies to assess gene expression levels in biological samples, including tumor tissues, thereby enhancing diagnostic accuracy and precision in clinical practice. Each gene within the diagnostic gene set described herein has been identified based on rigorous statistical analysis correlating gene expression data with clinical phenotypes, demonstrating significant associations with the disease's pathological features.

As used herein, the term ‘gene’ generally refers to the portion of a nucleic acid that encodes a protein. It should be understood that the term gene includes the coding sequence of a gene as well as gene regulatory sequences (e.g., promoters, enhancers, etc.) and/or intron sequences. It will further be appreciated that the definition of a gene includes reference to a nucleic acid that does not encode a protein but rather encodes a functional RNA molecule (e.g., an RNAi agent, a ribozyme, a tRNA, etc.). The term may optionally include control sequences, which will be apparent to the skilled person from the context.

The term ‘gene expression’ refers to the process by which genetic information encoded in a gene is converted into functional products such as proteins or RNA molecules. Gene expression normally occurs in two main stages: transcription, where the DNA sequence of a gene is transcribed into messenger RNA (mRNA), and translation, where the mRNA is translated into a protein. The term should be understood to be inclusive of transcription, translation, and any other specific processes regulating transcription and/or translation. Cancer cells often exhibit upregulated (increased) or downregulated (decreased) expression of key genes that regulate cell growth, division, and survival.

The term “surgery” includes curative surgery which removes a cancerous tumor or growth from the body of a subject, preferably used when the cancerous tumor is localized to a specific area of the body. Often used with radiation treatment before or after the surgery, Preventive surgery used to remove tissue that does not contain cancerous cells, but may develop into a malignant tumor. For example, polyps in the colon may be considered precancerous tissue and preventative surgery may be performed to remove them. Diagnostic surgery that determines whether cells are cancerous. Diagnostic surgery includes removing a tissue sample for testing and evaluation (in a laboratory by a pathologist) then confirming a diagnosis, identifying the type of cancer, and/or determining the stage of the cancer. Staging surgery used to uncover the extent of cancer, or the extent of the disease in the body and may include laparoscopy (i.e., a viewing tube with a lens or camera is inserted through a small incision to examine the inside of the body and to remove tissue samples). Debulking surgery to remove a portion, though not all, of a cancerous tumor when removing an entire tumor may cause damage to an organ or the body, Chemotherapy and/or radiation may be used after debulking surgery is performed. Palliative surgery used to treat cancer at advanced stages and relieve discomfort or to correct other problems cancer or cancer treatment may have created. Supportive surgery to work together with other cancer treatments, e.g., supportive surgery is the insertion of a catheter to help with chemotherapy. Cryosurgery uses extremely cold temperatures to kill cancer cells and is used most often with skin cancer and cervical cancer. Depending on whether the tumor is inside or outside the body, liquid nitrogen is placed on the skin or in an instrument called a cryoprobe (which is inserted into the body so that it touches the tumor). Laser surgery uses beams of light energy instead of instruments to remove very small cancers (without damaging surrounding tissue), to shrink or destroy tumors, or to activate drugs to kill cancer cells. Electrosurgery uses electrical current to kill cancer cells.

Aspects of the present disclosure are directed toward the utilization of gene expression data to identify breast cancer (BC) intrinsic subtypes, predict prognosis and treatment response, and direct and/or inform treatment choices. The present disclosure identifies biomarkers for basal-like breast cancer (BLBC), characterized by aggressive behavior and variability in therapeutic responses, and provides informed options for the treatment of basal-like breast cancer selected to provide better predicted treatment outcomes. This identification is achieved through whole-gene expression profiling combined with artificial intelligence techniques, facilitating an understanding of the biological characteristics of BLBC and enhancing diagnostic and therapeutic approaches.

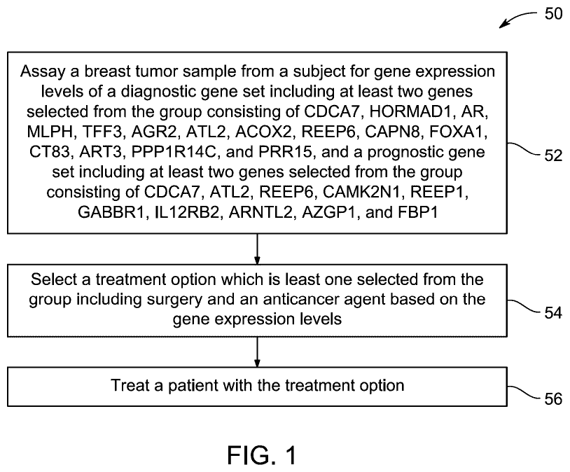

FIG. 1 illustrates a schematic flow chart of a method 50 for treating a breast cancer in a subject in need thereof. The order in which the method 50 is described is not intended to be construed as a limitation, and any number of the described method steps can be combined to implement the method 50. Additionally, individual steps may be removed or skipped from the method 50 without departing from the spirit and scope of the present disclosure.

At step 52, the method 50 includes assaying a breast tumor sample from the subject for gene expression levels of a diagnostic gene set and a prognostic gene set. In some embodiments, the diagnostic gene set includes at least two genes selected from CDCA7, HORMAD1, AR, MLPH, TFF3, AGR2, ATL2, ACOX2, REEP6, CAPN8, FOXA1, CT83, ART3, PPP1R14C, and PRR15. In some embodiments, the prognostic gene set includes at least two genes selected from the group consisting of CDCA7, ATL2, REEP6, CAMK2N1, REEP1, GABBR1, IL12RB2, ARNTL2, AZGP1, and FBP1.

In some embodiments, the method further includes comparing the gene expression levels of the diagnostic gene set and/or the prognostic gene set to a reference sample. In general, the reference sample can be a sample obtained from any suitable type and/or sub-type of cancer, such as breast cancer or basal-like breast cancer or triple-negative breast cancer. In some embodiments, the degree of variation in the gene expression levels between the breast tissue sample and the reference sample is indicative of the presence of cancer, the stage of cancer, the type of cancer, the sub-type of cancer, a prognosis of the cancer, or a combination of these. In some embodiments, a diagnosis of the cancer type and/or sub-type can be made based on the gene expression levels. In some embodiments, this diagnosis is made based on the gene expression levels of two or more genes in the diagnostic gene set. For example, a breast cancer sub-type may be determined based on the genes CDCA7, HORMAD1, and AR in the diagnostic gene set. Based on the expression levels of CDCA7, HORMAD1, and AR compared to one or more reference samples, the breast cancer can be determined to be a basal-like breast cancer.

In general, the gene expression level of any suitable gene can be determined by any suitable method known to one of ordinary skill in the art. Examples of suitable such methods and techniques include, but are not limited to northern blotting, quantitative PCR (qPCR), microarray analysis, RNA-seq, and combinations of these.

In northern blot analysis, the RNA is size fractionated, for example by polyacrylamide or agarose gel electrophoresis, and transferred to a nitrocellulose membrane. RNA molecules form covalent bonds with the nitrocellulose membrane. Probes that are complimentary to the transcript are labeled prior to hybridization with a chemiluminescence dye. The nitrocellulose membrane with the bound RNA is hybridized with one or more specifically labeled probes. The hybridized nitrocellulose membrane is then processed for detection of signal to determine the presence or the absence of the transcript. Northern blotting is often used to measure tRNA or a specific set of mRNAs.

qPCR is a method which can be used to quantify gene expression in real time. The expression level can be measured using a spectrophotometer, mRNA is often used as the template for the qPCR reaction. During qPCR, the mRNA template is converted to complimentary DNA (cDNA) using reverse transcriptase. Then, the single-stranded cDNA is synthesized into double-stranded DNA using DNA polymerase. A label (typically a fluorescent label) is added to the reaction mixture so that, as the amplified DNA molecules accumulate, the fluorescence values are recorded at the end of each cycle. The level of the fluorescent signal is directly proportional to the amount of amplified DNA that is present.

Microarray technology employs the principle of nucleic acid hybridization of cDNA strands to quantify a large number of transcripts in a single experiment. Two types of microarrays are typically used to measure gene expression. The in situ synthesized oligonucleotide microarray uses probe oligonucleotides that are 25 bases in length and are attached to a chip surface by a light-directed method. The eDNA microarray uses a single-stranded cDNA that is reverse transcribed from a single strand of mRNA. The single-stranded cDNA is converted into double-stranded DNA through PCR amplification. Then, the PCR product is immobilized on the array as the probe. RNA is extracted from a tissue and reverse transcribed to cDNA. Then, cDNA is processed for labeling. The labeled nucleic acids are transferred to a microarray chip for hybridization with immobilized probes. The microarray chips are hybridized then washed. The hybridized, tagged, fluorescent-labeled nucleic acid sequences remain on the microarray chip. Then, the hybridized microarray chip is scanned to read the fluorescent excitation signals. The intensity of the fluorescent signals detected is directly proportional to the amount of transcribed RNA.

RNA-Seq quantifies the levels of various types of RNA in a sample by sequencing the RNA directly and counting the number of sequences.

In some embodiments, the method may include using RNA sequencing technology to measure the expression levels of the diagnostic gene set and/or the prognostic gene set. In some embodiments, the method may include multiplex assays. In some embodiments, the gene expression levels of the diagnostic gene set and the prognostic gene set may be measured using the same technique. In some embodiments, the gene expression levels of the diagnostic gene set may be measured using a first technique and the gene expression levels of the prognostic gene set may be measured using a second technique. That is, the gene expression levels of the diagnostic gene set and the prognostic gene set are measured using different techniques.

In some embodiments, the method may utilize bioinformatics tools to analyze the gene expression data to identify patterns linked to breast cancer subtypes. This analysis may involve machine learning algorithms that categorize the tumor sample into a specific cancer type and/or sub-type (for example, breast cancer can be classified as either basal-like or non-basal-like) based on its gene expression profile. In some embodiments, the method may correlate the expression levels of genes in the prognostic gene set with clinical outcomes, such as relapse-free survival (RFS) and overall survival (OS), where high expression of certain genes may indicate a prognosis and inform treatment decisions. In some embodiments, the method may include validation steps with independent datasets to confirm the expression levels of the diagnostic and prognostic gene sets in additional breast tumor samples from patient populations, ensuring the applicability of the assay. Any identified genes may be added to the diagnostic gene set and/or prognostic gene set as appropriate.

At step 54, the method 50 includes selecting a treatment option which is least one selected from the group including surgery and an anticancer agent based on the gene expression levels. In some embodiments, the method may include selecting a surgical treatment, such as a lumpectomy or mastectomy, based on the characteristics of the tumor as indicated by gene expression levels. In some embodiments, the method may include choosing a specific anticancer agent, like a chemotherapy drug or targeted therapy, that is identified as effective according to the diagnostic and/or prognostic gene sets. In some embodiments, the selection process may use clinical decision support systems to analyze the gene expression data alongside other specific data, such as individual patient factors, to enhance treatment options. In some embodiments, the method may prioritize treatment choices that have demonstrated efficacy in clinical trials for similar gene expression profiles. Moreover, in some embodiments, the method may allow for the identification and/or selection of combination therapies that integrate multiple treatment agents (such as combinations of anti-cancer agents) and/or types (such as chemotherapy and immunotherapy), based on the analysis of gene expression data to improve treatment outcomes.

At step 56, the method 50 includes treating the patient with the treatment option. In some embodiments, the method may include performing a surgical procedure based on the diagnostic findings. In some embodiments, the surgical procedure includes a procedure to remove the tumor. In general, the surgical procedure can be any suitable surgical procedure known to one of ordinary skill in the art. For example, for breast cancer, typical surgical procedures include a lumpectomy or a mastectomy depending on the tumor's size and stage.

In some embodiments, the method may include administering an anticancer agent. In general, any anticancer agent or combination of anticancer agents can be used. In some embodiment, the anticancer agent is selected based on the gene expression level of a gene in the diagnostic set and/or prognostic set. For example, the anticancer agent can be a tailored chemotherapy regimen identified through gene expression levels. In some embodiments, the method may utilize targeted therapies that inhibit the action of proteins produced by overexpressed genes, such as androgen receptor inhibitors for high AR expression. In some embodiments, immunotherapy may be employed to enhance the patient's immune response against the tumor using agents like checkpoint inhibitors or monoclonal antibodies. In some embodiments, the method may include a combination of treatment options, such as concurrent chemotherapy and immunotherapy, based on the gene expression profile to enhance efficacy. In some embodiments, a personalized treatment plan may be developed that integrates gene expression results with other clinically relevant data, such as the patient's clinical history and preferences. In some embodiments, a monitoring protocol may be established to assess the patient's response to treatment through follow-up imaging and gene expression analyses. Such a monitoring protocol may be advantageous for allowing for necessary adjustments. In some embodiments, the method may include administering adjuvant therapy, such as hormone therapy or additional chemotherapy, after the initial treatment to minimize recurrence risk based on the prognostic gene set analysis.

In some embodiments, the anticancer agent is at least one selected from the group consisting of an anticancer agent targeting MSLN, an anticancer agent targeting TFF1, an anticancer agent targeting KRT16, an anticancer agent targeting IMPA2, an anticancer agent targeting FBPI, an anticancer agent targeting AR. In some embodiments, the anticancer agent targeting MSLN is amatuximab. In some embodiments, the anticancer agents targeting MSLN may include, but is not limited to, monoclonal antibodies such as MORAb-009 and DS-8201a; small molecule inhibitors that inhibit MSLN expression or it signaling pathways.

In some embodiments, the anticancer agent targeting TFF1 is at least one selected from the group consisting of raloxifene and afimoxifene. In some embodiments, the anticancer agents targeting TFF1 may be selected from groups such as including estrogen receptor modulators such as tamoxifen, small molecule inhibitors targeting pathways such as PI3K or AKT, and monoclonal antibodies designed for TFF1. In some embodiments, the anticancer agent is selected based on the gene expression levels of the prognostic gene set.

In some embodiments, the anticancer agent targeting KRT16 is at least one selected from the group consisting of zinc, zinc acetate, and zinc chloride. In some embodiments, the anticancer agents targeting KRT16 may include, but is not limited to, small molecule inhibitors including HDAC inhibitors such as vorinostat and panobinostat; proteasome inhibitors such as bortezomib; biologies such as monoclonal; RNA interference agents, such as siRNA or shRNA targeting KRT16.

In some embodiments, the anticancer agent targeting IMPA2 is at least one selected from the group consisting of lithium cation, lithium citrate, lithium succinate, and lithium carbonate. In some embodiments, the anticancer agent targeting IMPA2 may include, but is not limited to, small molecule inhibitors specifically designed to inhibit IMPA2 activity; monoclonal antibodies targeting IMPA2 or related pathways, RNA interference agents such as siRNA or shRNA to reduce IMPA2 expression; antisense oligonucleotides.

In some embodiments, the anticancer agent targeting FBP1 is at least one selected from the group consisting of adenosine phosphate, Mdl-29951, fructose-6-phosphate, MB-07803, and managlinat dialanetil. In some embodiments, the anticancer agent targeting FBP1 may include, but is not limited to, small molecule inhibitors specifically designed to inhibit FBP1 activity; monoclonal antibodies that target FBP1 or related pathways; RNA interference agents such as SIRNA or shRNA to reduce FBP1 expression; antisense oligonucleotides aimed at inhibiting FBP1 expression.

In some embodiments, the anticancer agent targeting AR is at least one selected from the group consisting of diethylstilbestrol, levonorgestrel, progesterone, spironolactone, flutamide, oxandrolone, and fluphenazine. In some embodiments, the anticancer agent targeting AR may include, but is not limited to, antiandrogens such as enzalutamide, abiraterone, bicalutamide; selective estrogen receptor modulators (SERMs) such as tamoxifen; aromatase inhibitors such as anastrozole, letrozole; corticosteroids such as dexamethasone; hormonal agents such as testosterone derivatives with modified activity; synthetic progestins such as norethisterone, medroxyprogesterone acetate.

In some embodiments, the diagnostic gene set includes at least three genes, preferably at least four genes, preferably at least five genes, preferably at least six genes, preferably at least seven genes, preferably at least eight genes, preferably at least nine genes, preferably at least ten genes, preferably at least eleven genes, preferably at least twelve genes, preferably at least thirteen genes, preferably at least fourteen genes selected from the group of CDCA7, HORMAD1, AR, MLPH, TFF3, AGR2, ATL2, ACOX2, REEP6, CAPN8, FOXA1, CT83, ART3, PPP1R14C, and PRR15. In some embodiments, the diagnostic gene set includes all of CDCA7, HORMAD1, AR, MLPH, TFF3, AGR2, ATL2, ACOX2, REEP6, CAPN8, FOXA1, CT83, ART3, PPP1R14C, and PRR15. In some embodiments, the diagnostic gene set may further include one or more genes selected from HER2, TP53, BRCA1, BRCA2, PIK3CA, EGFR, K167, PTEN, CCND1, FGFR1, VEGF.

In some embodiments, the prognostic gene set includes at least three genes, preferably at least four genes, preferably at least five genes, preferably at least six genes, preferably at least seven genes, preferably at least eight genes, preferably at least nine genes selected from the group of CDCA7, ATL2, REEP6, CAMK2N1, REEP1, GABBR1, IL12RB2, ARNTL2, AZGP1, and FBP1. In some embodiments, the prognostic gene set includes all of CDCA7, ATL2, REEP6, CAMK2N1, REEP1, GABBR1, IL12RB2, ARNTL2, AZGP1, and FBP1. In some embodiments, the prognostic gene set may further include one or more genes selected from TP53, BRCA1, BRCA2, PIK3CA, HER2 (ERBB2), FOXA1, CCND1, MMPs, VEGF, Ki67, EGFR.

In general, the anti-cancer agent can be administered in any suitable form and by any suitable route. For example, the anti-cancer agent can be administered via oral administration, parenteral administration, rectal administration, topical administration, transdermal administration, intralesional administration, or inhalation administration. Depending on the intended mode of administration, the anti-cancer agent can be in the form of solid, semi-solid, or liquid dosage forms, such as, for example, tablets, suppositories, pills, capsules, powders, liquids, or suspensions, preferably in a unit dosage form suitable for single administration of a precise dosage.

As used herein, the term ‘oral administration’ refers to the most common routes of drug administration, where medications are taken by mouth and absorbed through the gastrointestinal tract. Oral medications can include tablets, capsules, syrups, or suspensions. They are convenient for patients and are often self-administered. However, oral medications may be subject to degradation in the gastrointestinal tract and may have variable absorption rates depending on factors such as gastric emptying and gastrointestinal pH.

As used herein, the term ‘parenteral administration’ refers to the route involving delivering medications directly into the body, bypassing the gastrointestinal tract. Parenteral administration can be achieved through various routes, including intravenous (IV), intramuscular (IM), subcutaneous (SC), or intradermal (ID) injection. These routes allow for rapid onset of action and precise control over drug delivery. Parenteral administration is often used for medications that require immediate effects, have poor oral bioavailability, or need to bypass the gastrointestinal tract due to issues such as nausea or vomiting.

As used herein, the term ‘rectal administration’ refers to administering medications by inserting them into the rectum using suppositories, enemas, or rectal solutions. This route is particularly useful for patients who cannot take medications orally or require local or systemic drug absorption. Rectal administration can provide rapid absorption and avoid issues associated with oral administration, such as first-pass metabolism. Common medications administered rectally include treatments for constipation, hemorrhoids, and inflammatory bowel disease.

Topical medications are applied directly to the skin or mucous membranes for localized or systemic effects. This route is commonly used for dermatological conditions, wound healing, pain management, and local anesthesia. Topical formulations include creams, ointments, gels, lotions, patches, and sprays. Topical administration allows for targeted drug delivery to specific areas while minimizing systemic side effects.

Transdermal delivery involves applying medications to the skin for systemic absorption. Unlike topical administration, transdermal medications are designed to penetrate the skin and enter the bloodstream, providing sustained drug release over an extended period. Transdermal patches are a common form of transdermal delivery and are used for medications such as hormonal contraceptives, nicotine replacement therapy, and pain management.

Intralesional administration involves injecting medications directly into a specific lesion or localized area, such as a tumor, cyst, or inflamed tissue. These injections allow for targeted delivery of medications to the site of action, minimizing systemic exposure and side effects. This route is commonly used in dermatology, oncology, and rheumatology to treat skin disorders, cancerous lesions, and joint inflammation.

Inhalation administration involves delivering medications directly into the respiratory tract for local or systemic effects. This route can be achieved through various devices such as metered-dose inhalers, dry powder inhalers, nebulizers, or vaporizers. It is commonly used for treating respiratory conditions such as asthma, chronic obstructive pulmonary disease (COPD), and respiratory infections. Inhalation allows for rapid absorption of medications into the lungs and bloodstream, resulting in quick onset of action and reduced systemic side effects.

In general, the administration of the anti-cancer agent can be consistent with the standards of medical practice, governmental agency approval or guidelines, professional medical opinion, or a combination of these. For the anti-cancer agent, the dosage and treatment duration are dependent on factors such as the bioavailability of a drug, administration mode, toxicity of a drug, gender, age, lifestyle, body weight, the use of other drugs and dietary supplements, cancer stage, tolerance, and resistance of the body to the administered drug, etc. The dosage and treatment can take such factors into account and adjusted accordingly. The anti-cancer agent may be administered in a single or multiple individual divided doses. In some embodiments, the interval of time between the administration of the anti-cancer and the administration of one or more additional therapies or treatments may be about 1-5 minutes, 1-30 minutes, 30 minutes to 60 minutes, 1 hour, 1-2 hours, 2-6 hours, 2-12 hours, 12-24 hours, 1-2 days, 2 days, 3 days, 4 days, 5 days, 6 days, 7 days, 1 week, 2 weeks, 3 weeks, 4 weeks, 5 weeks, 6 weeks, 7 weeks, 8 weeks, 9 weeks, 10 weeks, 15 weeks, 20 weeks, 26 weeks, 52 weeks, 11-15 weeks, 15-20 weeks, 20-30 weeks, 30-40 weeks, 40-50 weeks, 1 month, 2 months, 3 months, 4 months, 5 months, 6 months, 7 months, 8 months, 9 months, 10 months, 11 months, 12 months, 1 year, 2 years, or any period in between. In certain embodiments, the anti-cancer agent and additional therapy and/or treatment are administered in less than 1 day, 1 week, 2 weeks, 3 weeks, 4 weeks, one month, 2 months, 3 months, 6 months, 1 year, 2 years, or 5 years apart.

### EXAMPLES

The following examples demonstrate a method for treating a breast cancer in a subject in need thereof. The examples are provided solely for illustration and are not to be construed as limitations of the present disclosure, as many variations thereof are possible without departing from the spirit and scope of the present disclosure.

### Example 1. Materials and Methods

A comprehensive stepwise approach was utilized to analyze transcriptomic data, employing bioinformatics and machine learning techniques to identify gene signatures that distinguish breast cancer patients into specific subtypes, specifically basal-like versus non-basal-like, and further classify basal-like patients based on favorable and unfavorable outcomes. A large cohort of 1,279 breast tumor gene expression profiles was assembled, with documented pre-classified subtypes. Differential expression analysis revealed 61 hub genes closely associated with basal-like breast cancer. Through machine learning methods, a novel 15-gene signature for diagnosing basal-like breast cancer was discovered, supported by hierarchical clustering and principal component analysis (PCA) that illustrated distinct groupings between the two subtypes. Furthermore, a prognostic 10-gene signature was identified, correlating significantly with both relapse-free survival and overall survival. A therapeutic 6-gene signature was also established, alongside a list of FDA-approved drugs potentially suitable for basal-like breast cancer (BLBC) and triple-negative breast cancer (TNBC). The robustness of the machine learning model was confirmed through ten-fold cross-validation on the same sample set (internal validation), external validation with an independent dataset of 351 samples, and additional validation using quantitative PCR (qPCR) to ensure platform-independent reliability.

### Example 2: Data Retrieval, Setup, and Preprocessing

The breast cancer (BC) gene expression dataset utilized in this study was sourced from the Gene Expression Omnibus for high-throughput functional genomic analysis. Twelve datasets corresponding to the GPL570 platform (Affymetrix Human Genome U133 Plus 2.0 Array) and probe ID 54675 were included: GSE10780, GSE111662 (Santucci-Pereira, J., et. al., Breast Cancer Research, 2019, 21(1), incorporated herein by reference in its entirety), GSE162228 (Chen, Y.-J., et. al., Bioscience Reports, 2021, 41(8), incorporated herein by reference in its entirety), GSE17907 (Sircoulomb, F., BMC Cancer, 2010, 10(1), incorporated herein by reference in its entirety). GSE20711 (Dedeurwaerder, S., et. al., EMBO Molecular Medicine, 2011, 3(12), 726-741, incorporated herein by reference in its entirety), GSE21653 (Sabatier, R., et. al., PLoS ONE, 6(11), 2011, incorporated herein by reference in its entirety), GSE30010, GSE45827 (Gruosso, T., et. al, EMBO Molecular Medicine, 2016, 8(5), 527-549, incorporated herein by reference in its entirety), GSE48390 (Huang, C. C., et. al., PLoS ONE, 2013, 8(10), incorporated herein by reference in its entirety), GSE58792 (Shike, M., et. al., JNCI Journal of the National Cancer Institute, 2014, 106(9), incorporated herein by reference in its entirety), GSE65194 (Maire, V., et. al., Cancer Research, 2013, 73(2), 813-823, incorporated herein by reference in its entirety), and GSE87007 (Raspé, E., et. al., EMBO Molecular Medicine, 2017, 9(8), 1052-1066, incorporated herein by reference in its entirety). These datasets were extracted using the ‘GEOquery’ library in R. Only datasets containing pre-classified intrinsic subtypes of BC and corresponding normal breast tissues were included. Samples lacking molecular subtype information or representing BC cell lines were excluded from the analysis.

Raw CEL files (n=1,279) from the twelve datasets were combined, followed by application of the robust multichip average (RMA) preprocessing method for background correction and normalization utilizing the affy package from R/Bioconductor. Batch effects were mitigated using the COMBAT algorithm within R 4.2.3. Probes were mapped to corresponding genes, and the average value for multiple probes associated with a single gene was calculated. Principal component analysis (PCA) was executed using the ‘prcomp’ function, while hierarchical clustering was performed with the ‘pheatmap’ package in R to assess correlations among samples and gene symbols. The resultant dataset of 1,279 samples was employed for training and testing the model using ten-fold cross-validation. An external validation dataset consisting of 351 BC patients was derived from two independent datasets: GSE31448 (Sabatier, R., et. al., PLoS ONE, 2011, 6(11), incorporated herein by reference in its entirety) and GSE43358.

### Example 3: Identification of Differentially Expressed Genes

Differentially expressed genes (DEGs) between the five subtypes of BC (Basal-like, HER2, Luminal A, Luminal B, and Normal-like) and normal non-tumor samples were identified using the LIMMA package in R 4.0. The filtration criteria employed were |log 2 fold change|>1 and p<0.05. Five DEG lists were generated by contrasting Basal-like samples with each of the other subtypes (HER2, Luminal A, Luminal B, Normal-like BC) and normal breast tissues. A Venn diagram was constructed using the ‘jvenn’ tool to ascertain the common and specific DEGs across the subtypes.

### Example 4: Machine Learning Methods for Screening of Gene Signatures

In the present disclosure, five machine learning (ML) techniques were employed to screen for diagnostically significant genes: Support Vector Machine (SVM), L1 Regularized SVM, Random Forest, Extremely Randomized Trees, and XGBoost. A voting method was utilized, where multiple individual models contributed to the decision-making process, with the final classification determined by the majority vote supported by the maximum ML results. The dataset consisted of 1,279 breast cancer (BC) patients and over 31,000 unique features, characterized by a high-dimensional feature space, which was particularly advantageous for identifying significant genes through ML techniques, resulting in robust model performance. The model was structured as a binary classification task using the development dataset, where gene expression values served as input variables, and the output variables corresponded to the classification of basal-like versus non-basal-like breast cancer (including HER2, Luminal A, Luminal B, normal-like, and normal).

### Example 5: Support Vector Machine (SVM)

SVM was employed as a powerful tool for managing high-dimensional data and capturing non-linear relationships. This technique was utilized to identify a subset of features that significantly distinguish between different breast cancer classes/groups. SVM operates by finding a hyperplane in the feature space that separates the classes, with the objective of maximizing the margin—the distance between the hyperplane and the nearest data point of any class, referred to as support vectors. This approach enables effective classification and enhances the interpretability of gene signatures relevant to breast cancer subtyping.

For a linearly separable case, the decision function for SVM can be represented using Equation 1 as

$$f ( x ) = w x + b \qquad ( 1 )$$

where ƒ(x) is the decision function, w is the weight vector, x is the input vector, and b is the bias term.

SVM minimizes the weight vector's norm (∥w∥) for accurate classification The optimization problem (Equation 2) is often formulated as

$${min}_{w , b} \frac{1}{2} {‖ w ‖}^{2} \qquad ( 2 )$$

subject to yᵢ(w·xᵢ+b)≥1 for all training samples (xᵢ,yᵢ), where yᵢ class label of xᵢ.

In non-perfectly separable data, SVM uses a margin, allowing some misclassification by introducing slack variables (ξ) into the optimization problem (Equation 3).

$${min}_{w , b , ξ} \frac{1}{2} {‖ w ‖}^{2} + C {\sum }_{i = 1}^{N} {ξ}_{i} \qquad ( 3 )$$

$$subject to {y}_{i} ( w . {x}_{i} + b ) \geq 1 - {ξ}_{t} and {ξ}_{i} \geq 0 .$$

### Example 6: L1 Regularized Support Vector Machine (SVM)

L1 Regularized Support Vector Machine (SVM) was utilized to facilitate critical feature selection by promoting sparsity within the feature space. This approach involves minimizing an objective function that includes a standard SVM loss term, which aims to achieve accurate classification, along with an L1 penalty term that imposes a cost on the absolute values of the feature weights. Incorporating this L1 penalty effectively reduces the number of features utilized in the model, thereby enhancing interpretability and focusing on the most significant variables for distinguishing between breast cancer subtypes.

### Example 7: Random Forest

Random Forest (RF), an ensemble learning algorithm characterized by its versatility and robustness, was employed to aggregate predictions from multiple randomized decision tree models. This method was specifically applied for feature selection by assessing the degree to which the accuracy of the model diminishes when a particular feature is randomly permuted. Features that resulted in a substantial decrease in model accuracy were identified as important contributors. During the training phase, RF constructed a multitude of decision trees, thus enabling comprehensive ranking of features based on their predictive significance. This methodology not only aids in feature selection but also enhances the overall predictive capability of the model.

Each decision tree is built by recursively splitting the feature space. The Gini impurity reduction at node m is maximized, given by Equation 4:

$$i ( m ) = {\sum }_{c = 1}^{C} {P}_{mc} ( 1 - {P}_{mc} ) \qquad ( 4 )$$

Here, P_{mc} is the fraction of class C observations at node m, ensuring leaf nodes' class purity.

Predictions are made by aggregated voting of all decision trees using Equation 5:

$$y = arg {max}_{c} {\sum }_{t = 1}^{T} / ( {y}_{t} = c ) \qquad ( 5 )$$

where, T represents the number of trees, yᵢ is the label predicted by tree t, and I( ) as an indicator function.

### Example 8: Extremely Randomized Trees

Extremely Randomized Trees (ExtraTrees), an ensemble learning algorithm that builds upon the principles of Random Forest, was employed for a robust feature selection process. This methodology involves selecting random thresholds for each feature, thereby introducing an additional layer of diversity in the model. Feature importance scores were calculated based on the decrease in impurity attributable to each feature across all decision trees within the ensemble. Features that contributed to a reduction in impurity during the tree-building process were identified as significant and deemed important for the classification task.

### Example 9: Xtreme Gradient Boosting (XGBoost)

extreme Gradient Boosting (XGBoost), a gradient-boosting algorithm recognized for its scalability, efficiency, and ability to manage diverse datasets, was utilized to regulate model complexity and mitigate the risk of overfitting. The algorithm effectively handles imbalanced datasets and incorporates both L1 (Lasso) and L2 (Ridge) regularization techniques to achieve a balance between model complexity and predictive accuracy. Feature importance scores in XGBoost were derived from metrics such as gain (improvement in predictive accuracy), coverage (relative quantity of observations), and frequency (relative occurrence of a feature across trees within the ensemble).

### Example 10: Survival Study

Survival analysis was conducted to evaluate time-to-event data, specifically focusing on endpoints such as time until death or disease progression. The Kaplan-Meier method was employed, incorporating hazard ratios, confidence intervals, and log-rank p-values, to ascertain associations between gene expression profiles and patient prognoses concerning disease progression and mortality outcomes. This analytical approach provided valuable insights into the relationship between molecular characteristics and clinical outcomes in breast cancer patients.

### Example 11: Hazard Function and Hazard Ratio

The hazard function represents the instantaneous potential for death at a specific time point, given survival up to that time. The survival function can be shown as Equation 6:

$$h ( t ) = {lim}_{Δ t \to 0} \frac{P [ death ( r , t + Δ t ) ]}{Δ t} \qquad ( 6 )$$

where death (t,t+Δt) refers to a person's passing between the ages of t and t+Δt within that time period.

$$H ( t ) = - {log}_{{e}^{s}} ( t ) \qquad ( 7 )$$

The survival function gives the probability of surviving past time t (Equation 7). These metrics can be used to compare survival between up and down expressed genes, often by calculating a hazard ratio (HR). The HR value in survival analysis of BC was used for quantitative comparison of prognosis between subgroups (up and down gene expressions) and identification of genes impacting outcomes. An HR above or below 1.0 indicate elevated risk or poorer outcome while an HR=1 means no difference in risk. Similarly, an HR value 2 indicates twice the higher risk of an event like death or recurrence between two groups.

### Example 12: Confidence Interval (CI)

The Kaplan-Meier survival curve provides point estimates of survival probabilities at given time points, but determining the precision of these estimates is also important. The CI was used to quantify the reliability of estimated survival probabilities over time and enable appropriate interpretation of survival findings. In the Kaplan-Meier approach, 95% CIs for the survival probabilities were calculated, which accounts for censoring of observations. Wider CIs indicate less precision, while narrower CIs reflect more precision.

### Example 13: Kaplan-Meier Method

The Kaplan-Meier estimator, a non-parametric method, was used to generate survival curves and calculate survival probabilities over time. It determined the probability of an event (such as death) occurring at a given time point, based on the proportion of study subjects surviving up to that point. The survival function (Equation 8) was calculated using the following formula:

$$SF ( Survival Function ) at t = {\prod }_{i = {t}_{i} < t} \frac{{n}_{i} - {d}_{i}}{{n}_{i}} = {\prod }_{i = {t}_{i} < t} ( 1 - {d}_{i} / {n}_{i} ) \qquad ( 8 )$$

When two events occur, the survival curve does not change, in tᵢ and tᵢ+1. recursive formula (Equation 9)

$$SF at {t}_{j} = [ \frac{{n}_{j - 1} - {d}_{j - 1}}{{n}_{j - 1}} ] multiply by ( SF ) at {t}_{j - 2} \qquad ( 9 )$$

Kaplan-Meier Plotter was used to visualize and compare survival outcomes associated with particular gene expression data from BC studies. The tool automatically determined the optimal cut-off value, and all datasets on the website were chosen for analysis based on the Basal-like subtype. Log-rank p-values<0.05 and hazard ratios>1< were cutoff used for significant genes.

### Example 14: Drug Target Identification

Initially, differentially expressed genes (DEGs) were selected based on their translation to proteins possessing valid UniProt identifiers. Subsequently, the presence of these proteins was validated as drug targets within the DrugBank database, specifically in relation to known FDA-approved pharmaceuticals.

### Example 15: Validation and Evaluation Metrics of Machine Learning Models

Machine learning model validation was conducted using a dual approach: internally via ten-fold cross-validation and externally through independent datasets utilizing various ML algorithms, namely Logistic Regression, Random Forest, SVM, Neural Network, Gradient Boosting, Extra Trees Classifier, XGB Classifier, and K-Nearest Neighbor Classifier. Performance metrics for the model were computed through ten-fold cross-validation, where the dataset was divided into ten subsets, and the model was trained and evaluated ten times. In each iteration, one of the ten subsets served as the test set for evaluating performance metrics, while the remaining nine subsets were utilized as the training set. This process was repeated for each subset. To assess the effectiveness of the ML models, standard classification evaluation metrics were employed, including accuracy, precision, recall, F1 score, and area under the receiver operating characteristic curve (AUC-ROC). The ROC curve evaluates the relationship between sensitivity and specificity at various thresholds. AUC is directly proportional to accuracy; a larger curve area indicates higher prediction accuracy. The ‘sklearn’ and ‘matplotlib’ packages were utilized to plot the AUC-ROC curves. The formula used for the computation of performance metrics is provided below. The formula used for the computation of performance metrics is given in Scheme 1 below.

$$Scheme 1. Formulae used for the computation of performance metrics .$$

$$Accuracy = \frac{True Positives + True Negatives}{Total Number of Predictions}$$

$$Precision = \frac{True Positives}{True Positives + False Positives}$$

$$Recall = \frac{True Positives}{True Positives + False Negatives}$$

$$F 1 - measure = \frac{2 * Precision * Recall}{Precision + Recall}$$

### Example 16: Logistic Regression

The logistic regression ML algorithm was applied for training and validating the models, particularly for binary classification tasks, because of its simplicity and interpretability. The logistic function, also known as the sigmoid function, maps a linear combination of input features to a probability between 0 and 1. The logistic function (Equation 10) can be written as

$$P ( y = 1 \vert X ) = \frac{1}{e - ( {β}_{0} + {β}_{1} {x}_{1} + {β}_{2} {x}_{2} + … + {β}_{n} {x}_{n} )} \qquad ( 10 )$$

where P(y=1|X) is the probability of the basal-like being 1 given the input feature X. β₀ is the bias term, β₁, β₂ . . . .βₙ are the coefficients associated with the input features x₁, x₂ . . . , xₙ, respectively. e is the base of the natural logarithm.

The coefficients (βᵢ) provide insights into the impact of each feature on the log-odds of the positive class. The log-odds, or the logit transformation (Equation 11), is the natural algorithm of the odds ratio (probability of success divided by the probability of failure).

$$logit ( P ) = log ( \frac{p}{1 - p} ) = {β}_{0} + {β}_{1} {x}_{1} + {β}_{2} {x}_{2} + … + {β}_{n} {x}_{n} \qquad ( 11 )$$

Here, p is the probability of belonging to the basal-like class. The logistic regression model was trained by maximizing the positive log-likelihood or minimizing the negative log-likelihood (minimizing the logistic loss) function to measure the likelihood of the observed data in the model parameters. Log-likelihood loss function (Equation 12) is shown as

$$Log Likelihood = - {\sum }_{i = 1}^{N} [ {y}_{i} log ( {p}_{i} ) + ( 1 - {y}_{i} ) log ( 1 - {p}_{i} ) ] \qquad ( 12 )$$

Here, N is the number of samples, yᵢ is the true label, and pᵢ is the predicted probability.

### Example 17: Neural Network (NN)

The classification model employed in this study is a multi-layer perceptron (MLP) neural network. The input layer consists of nodes corresponding to each feature in the data. Connected to this, a hidden layer with 100 rectified linear unit (ReLU) activation nodes is employed (Equation 13). For given training data points (x₁,y₁), (x₂,y₂) . . . , (xₙ,yₙ)∈ [symbol] and yᵢ∈{0, 1}, one hidden layer one hidden neuron MLP learns the function

$$f ( x ) = {W}_{2} g ( {W}_{1}^{T} x + {b}_{1} ) + {b}_{2} \qquad ( 13 )$$

Where W₁∈ [symbol] and W₂, b₁, b₂∈ [symbol] are model parameters while g(x) represents a specific function of x. W₁ and W₂ represent the weights associated with the input layer and hidden layer, respectively, while b₁ and b₂ are the bias terms applied in the hidden and output layers.

The ReLU activation facilitates efficient backpropagation during training. The logistic function (Equation 14) transforms the output to probabilities between 0 and 1 for binary classification.

$$g ( z ) = \frac{1}{( 1 + {e}^{- z} )} \qquad ( 14 )$$

The Average Cross-Entropy loss for binary classification (Equation 15) is defined as,

$$Loss ( y , y , W ) = - \frac{1}{n} {\sum }_{i = 0}^{n} ( {y}_{i} ln {y}_{i} + ( 1 - {y}_{i )} l n ( 1 - {y}_{i} ) ) + \frac{a}{2 n} {‖ W ‖}_{2}^{2} \qquad ( 15 )$$

where

$$α {‖ W ‖}_{2}^{2}$$

is an L₂-regularize term to prevent model from overfitting; and α>0 is non-negation hyperparameter that controls the magnitude of the penalty. The Adam algorithm is employed for efficient gradient-based optimization.

### Example 18: Gradient Boosting

Gradient boosting is an ensemble ML technique that produces a strong predictive model by combining multiple weaker models, typically decision trees. It builds the ensemble sequentially, with each new model attempting to correct its predecessor's errors.

### Example 19: K-Nearest Neighbors Classifier (kNN)

kNN is a popular machine learning algorithm belonging to the family of instance-based or lazy learning algorithms, which means that it does not attempt to learn a function from the training data. Instead, kNN stores all the training data and classifies new data based on the similarity of its features to those in the training set. The number of nearest neighbors (K) is a hyperparameter that can be tuned to improve performance. For training data points D given by Equation 16, for a point x∈χ^{d}, the set of k neighbors is defined Sₓ⊆D such that |Sₓ|=k and for all

$$x ⊆ \frac{D}{{S}_{x}} .$$

$$D = \{ ( {x}_{1} , {y}_{1} ) , ( {x}_{1} , {y}_{1} ) , … , ( {x}_{1} , {y}_{1} ) \} ⊆ {χ}^{d} X y \qquad ( 16 )$$

The function ‘dist’ (Equation 17) computes the distance between two points in χ^{d}, a set Sₓ of size k can be defined as

$$dist ( x , {x}^{′} ) \geq {max}_{( x , y ) ∈ {S}_{x}} dist ( x , {x}^{′} ) , ∀ ( {x}^{′} , {y}^{′} ) ∈ \frac{D}{{S}_{x}} . \qquad ( 17 )$$

That is every point in D but not in Sₓ is at least as far away from x as the furthest point in Sₓ. The classifier function (Equation 18) outputs the most frequent label among the set of nearest neighbors Sₓ for data point x.

$$h ( x ) = mode ( \{ y : ( {x}^{′} , {y}^{′} ) ∈ {S}_{x} \} ) \qquad ( 18 )$$

The effectiveness of a k-nearest neighbor classifier stems directly from how well its distance metric captures similarity with respect to class labels. This enables the model to select neighbors that accurately predict the label of a query point. The Minkowski distance (Equation 19) is typically the preferred choice.

$$d i s t ( x , {x}^{′} ) = {‖ x - {x}^{′} ‖}_{∞} = {( {{Σ}_{i = 1}^{d} ( \vert {x}_{i} - {x}_{i}^{′} )}^{p} )}^{\frac{1}{p}} , p \geq 1 \qquad ( 19 )$$

### Example 19: Validation at RNA and Protein Expression

UALCAN (The University of Alabama at Birmingham CANcer) portal is an interactive web resource for analyzing cancer OMICS data. It has RNA expression, protein expression, methylation, and survival data from publicly available cancer databases such as TCGA (The Cancer Genome Atlas Program) and CPTAC (Clinical Proteomic Tumor Analysis Consortium). RNA-seq data from the TCGA database and protein expression data from the CPTAC database were used for validation at the RNA and protein levels.

### Example 20: Real-Time Quantitative PCR

Expression of HORMAD1, CT83, PPP1R14C, CDCA7, ART3, ATL2, ARNTL2, GABBR1, IL12RB2, FOXA1, AGR2, TFF3, MLPH, AR, PRR15, FBP1, CAPN8, ACOX2, REEP6, REEP1, AZGP1, and CAMK2N1 genes was validated using a quantitative PCR (qPCR) assay conducted on the Applied Biosystems StepOnePlus Real-Time PCR instrument (ThermoFisher Scientific, USA). Quantification was performed using PowerUp™ SYBR™ Green Master Mix using GAPDH1 as reference. DataAssist™ Software were used for initial Ct values calculation and comparative Ct (ΔΔCt) method was used for quantitative gene expression. Additionally, RNA-seq results available online at the UALCAN portal (https://ualcan.path.uab.edu/index.html) were used to confirm the expression pattern of these genes at an independent bigger cohort of the TCGA dataset.

### Example 21: Statistical Analysis

All statistical analyses were done using SPSS 22.0 (SPSS, Inc.) and R 2.4.0. Differences between BC subtypes with regard to clinicopathological characteristics were examined using χ² tests and ANOVA. Univariate survival curves were generated by the Kaplan-Meier method and differences in survival among the BC subtypes were assessed by the log-rank test. P value<0.05 was considered significant.

### Example 22: Differentially Expressed Genes in Basal-Like Subtype of BC

1279 BC samples including basal-like (19.15%), HER2-enriched (15.56%), luminal A (21.42%), luminal B (16.42%), normal-like (3.60%), and normal (23.85%) breast tissues from Affymetrix GeneChip Human Genome U133 Plus 2.0 arrays platform [HG-U133_Plus_2] with 54,675 features/probes and clinical information showing intrinsic subtypes from twelve BC GEO datasets (GSE20711, GSE87007, GSE21653, GSE17907, GSE48390, GSE45827, GSE65194, GSE162228, GSE58792, GSE111662, GSE30010, GSE10780) were used as discovery cohorts. Expression data was GC-RMA normalized and 46597 out of 54,675 hybridization probes passed the cutoff: median expression >5.5 and present in at least two samples. The ML model was developed and internally validated on same cohort while external validation was done on 351 BC samples from two independent datasets (GSE31448 and GSE43358) (Table 1).

TABLE 1
Datasets include different intrinsic subtypes of breast cancer and
normal breast tissue samples retrieved from GPL570 platform.

| | | | | | | | | | |
|---|---|---|---|---|---|---|---|---|---|
| | Original | Excluded | Analysed | Basal | HER2- | Luminal | Luminal | Normal- | |
| GEO ID | Samples | Samples | Samples | like | enriched | A | B | like | Normal |

Testing Datasets

| | | | | | | | | | |
|---|---|---|---|---|---|---|---|---|---|
| GSE20711 | 90 | 0 | 90 | 27 | 26 | 13 | 22 | 0 | 2 |
| GSE87007 | 31 | 0 | 31 | 10 | 7 | 5 | 9 | 0 | 0 |
| GSE21653 | 266 | 0 | 266 | 75 | 24 | 89 | 49 | 29 | 0 |
| GSE17907 | 55 | 3 | 52 | 1 | 35 | 8 | 0 | 4 | 4 |
| GSE48390 | 81 | 7 | 74 | 10 | 14 | 22 | 15 | 13 | 0 |
| GSE45827 | 155 | 14 | 141 | 41 | 30 | 29 | 30 | 0 | 11 |
| GSE65194 | 178 | 14 | 164 | 55 | 39 | 29 | 30 | 0 | 11 |
| GSE162228 | 133 | 0 | 133 | 17 | 22 | 54 | 40 | 0 | 0 |
| GSE58792 | 51 | 0 | 51 | 9 | 2 | 25 | 15 | 0 | 0 |
| GSE111662 | 27 | 0 | 27 | 0 | 0 | 0 | 0 | 0 | 27 |
| GSE30010 | 107 | 0 | 107 | 0 | 0 | 0 | 0 | 0 | 107 |
| GSE10780 | 143 | 0 | 143 | 0 | 0 | 0 | 0 | 0 | 143 |
| | 1317 | 38 | 1279 | 245 | 199 | 274 | 210 | 46 | 305 |

Validation Datasets

| | | | | | | | | | |
|---|---|---|---|---|---|---|---|---|---|
| GSE31448 | 357 | 63 | 294 | 98 | 26 | 90 | 49 | 0 | 31 |
| GSE43358 | 57 | 0 | 57 | 0 | 14 | 16 | 10 | 0 | 17 |
| | 414 | 63 | 351 | 98 | 40 | 106 | 59 | 0 | 48 |

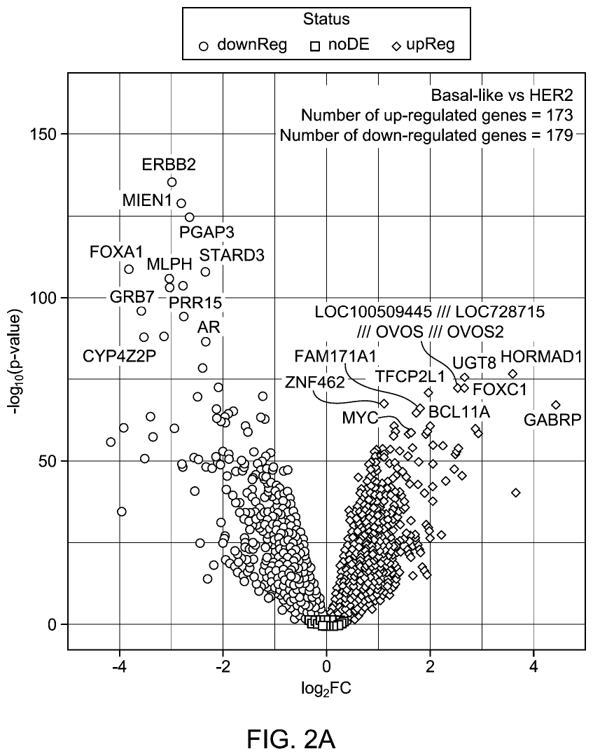

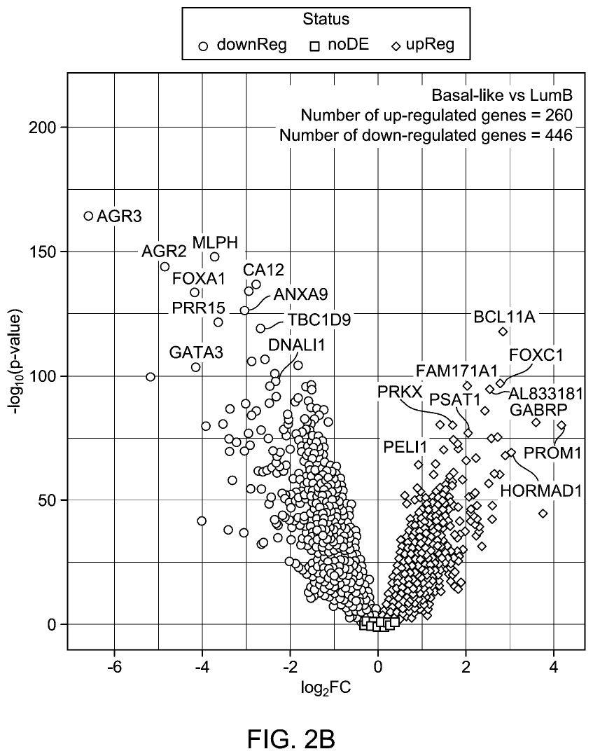

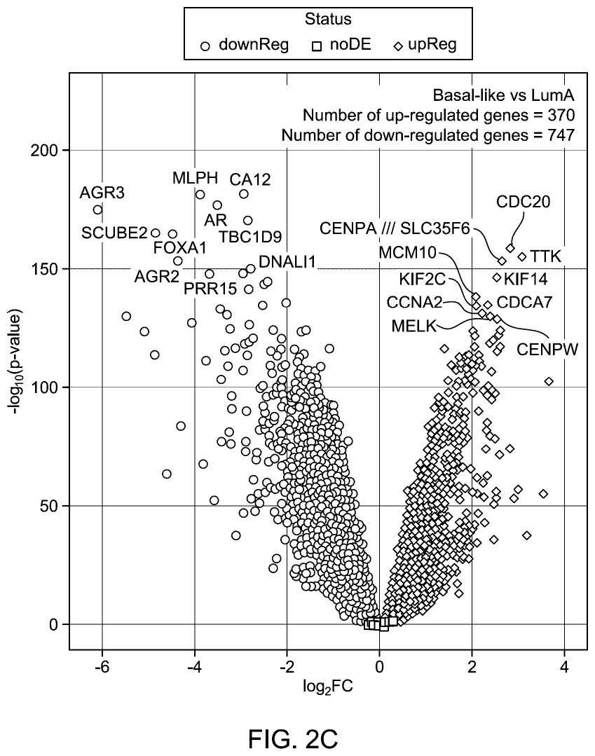

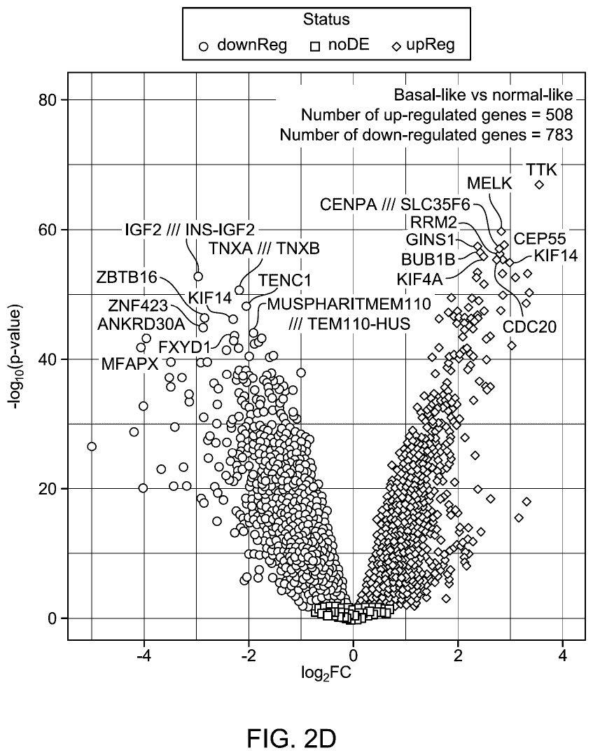

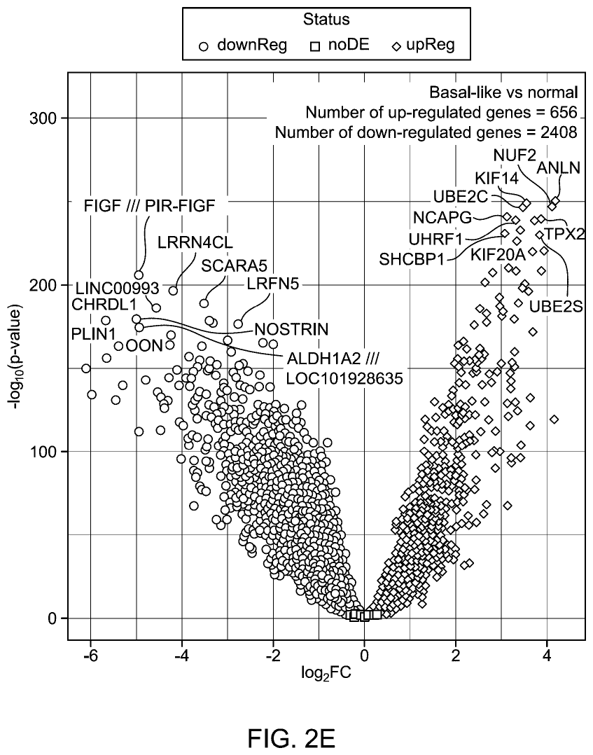

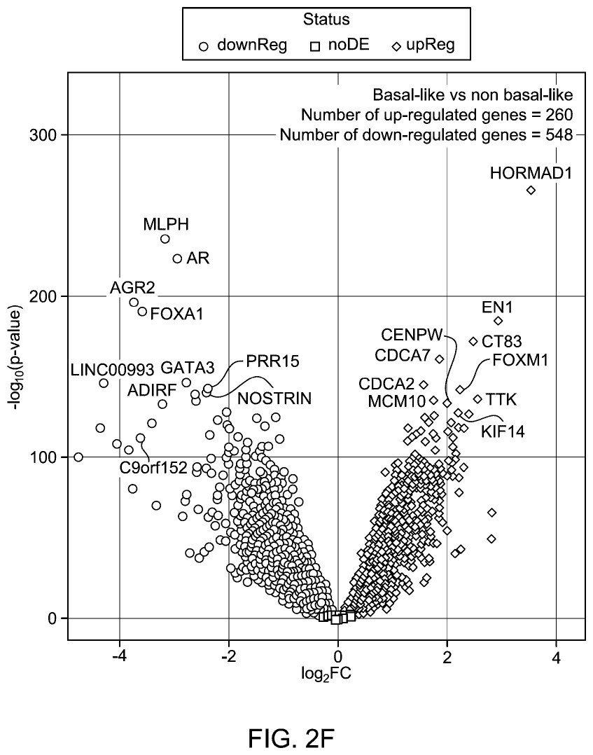

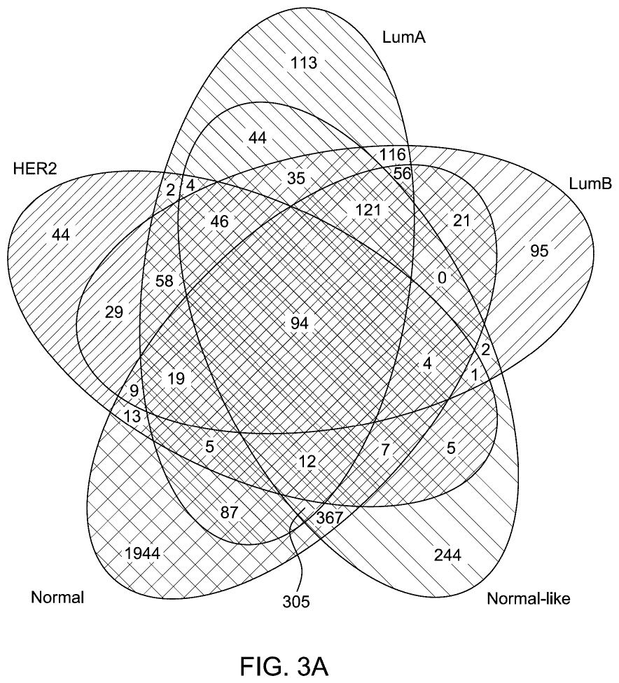

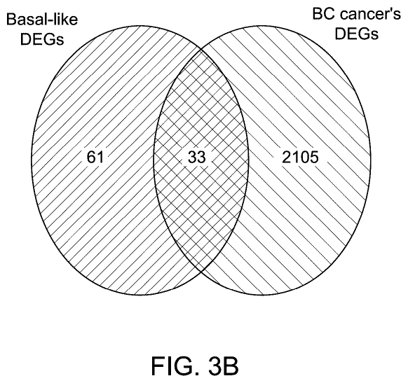

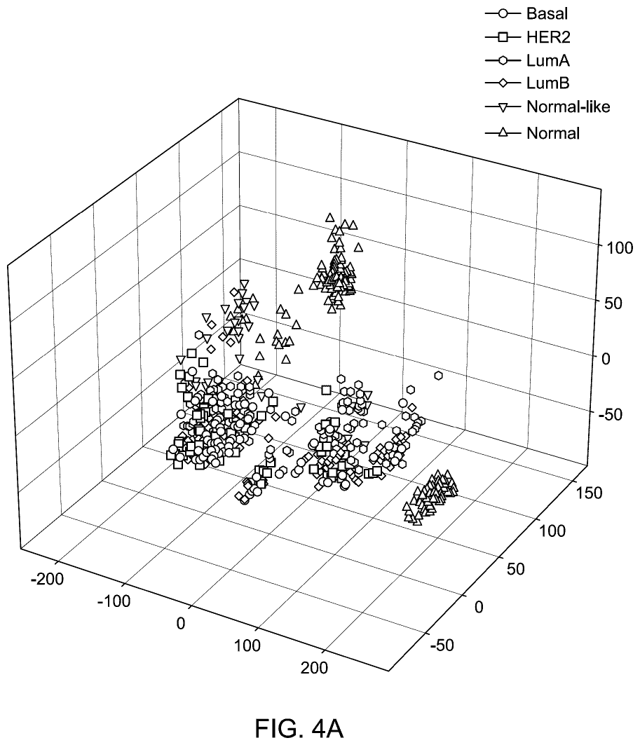

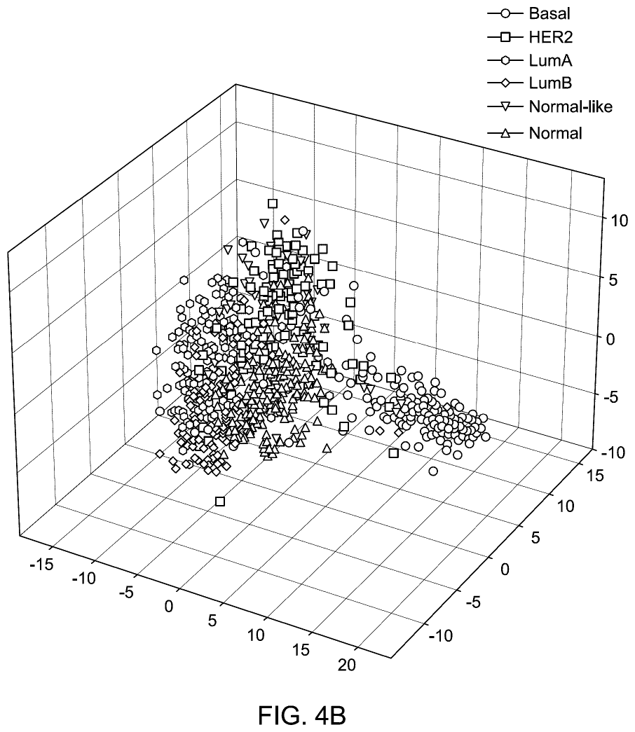

Multiple sets of DEGs as gene expression of basal-like was compared against HER2 (352 DEGs, 173-up and 179-down), LumB (706 DEGs, 260-up and 446 down), LumA (1117DEGs, 370-mp and 747-down); normal-like (1291 DEGs, 508-up and 783-down), normal (3064 DEGs, 656-up and 2408-down) and combined non-basal-like (808 DEGs, 260-up and 548-down) (FIGS. 2A-2F). Venn-diagram of DEGs from BC subtypes revealed 94 common genes (FIGS. 3A-3B). Additionally, 2138 DEGs, including 480 upregulated and 1658 downregulated, were identified for the BC cohort. Sixty-one basal-like associated genes were filtered, including ABCC11, ACOX2, ADIRF, AGR2, AGR3, AL833181, ANKRD30A, ANP32E, AR, ARNTL2, ART3, ATL2, AZGP1, C9orf152, CAMK2N1, CAPN8, CDCA7, CDKN2A, CPA3, CT83, CYP4Z1, CYP4Z2P, DACH1, DHRS2, DUSP4, FAM110C, FAM174B, FBP1, FOXA1, GABBR1, GATA3, HORMAD1, IL12RB2, IMPA2, KIAA1324, KIAA1804, KRT16, LIMA1, OVOS, LOC101060264, TMC5, MLPH, MSLN, MUC16, PPP1R14C, PRKX, PRR15, REEP1, REEP6, SERHL, SHISA2, SLC40A1, SMIM14, SYT17, TFAP2B, TFF1, TFF3, TMEM158, TOX3, ZG16B, and ZNF703, by excluding 33 genes that were present in all subtypes of cancer, including APOD, AZGP1, CADPS2, CDC20, CDCA2, CENPW, CPE, CYP4X1, EN1, FDCSP, FOXM1, GPD1L, LAMP3, LINC00993, MMP12, MUCL1, NDC80, NOSTRIN, NPY1R, NR2F1, NTN4, PIP, PRAME, PRICKLE2, S100A7, S100A8, S100A9, SCGB1D2, SCGB2A1, SCGB2A2, SCUBE2, SEMA3C, and TTK (Table 2). The principal component analysis (PCA) plot exhibited grouping of samples based on gene expression, with the 61 filtered genes clearly differentiating basal-like subtypes from other breast cancer subtypes as well as normal breast tissues (FIGS. 4A-4B).

TABLE 2
Average gene expression values for basal-like and non-basal-like subtypes.

| | Genes exclusive for basal-like | | | | Genes not specific to any BC subtypes | | | |
|---|---|---|---|---|---|---|---|---|
| Subtype/Genes | ABCC11 | ACOX2 | AGR3 | AR | APOD | CDCA2 | COL10A1 | ESRP1 |
| Basal-like | 4.517 | 5.747 | 4.709 | 5.574 | 7.806 | 6.928 | 8.277 | 9.932 |
| HER2 | 6.449 | 7.744 | 6.436 | 8.332 | 8.651 | 5.787 | 9.763 | 9.927 |
| LumB | 5.786 | 7.896 | 11.304 | 9.078 | 9.195 | 5.780 | 9.791 | 9.792 |
| LumA | 5.932 | 7.765 | 10.811 | 8.446 | 10.513 | 5.254 | 9.879 | 9.174 |
| Normal-like | 5.824 | 7.612 | 7.627 | 8.168 | 11.988 | 4.906 | 8.207 | 8.861 |
| Normal | 6.001 | 7.736 | 7.856 | 8.146 | 11.275 | 4.878 | 4.632 | 7.766 |

### Example 23: Machine Learning Algorithms for the Identification of Diagnostic Biomarker Genes

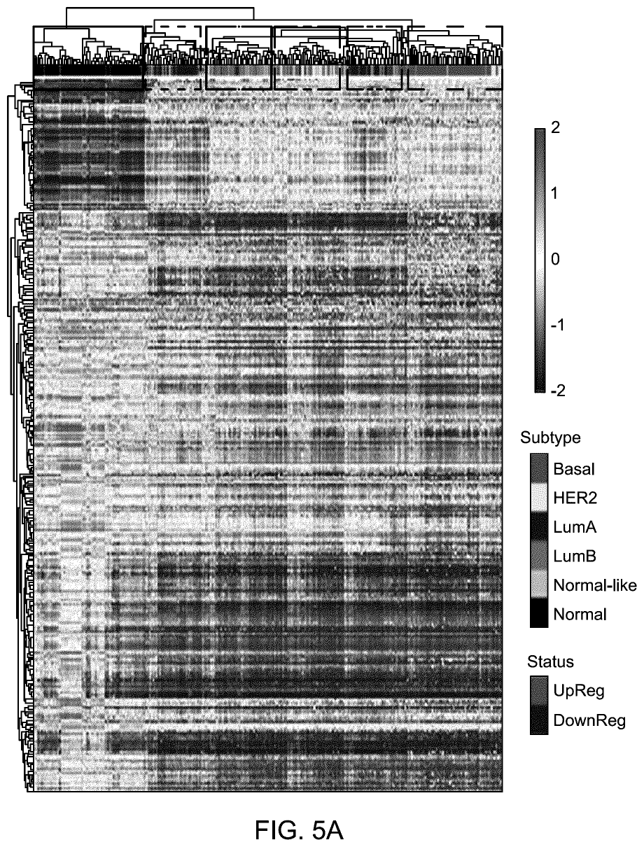

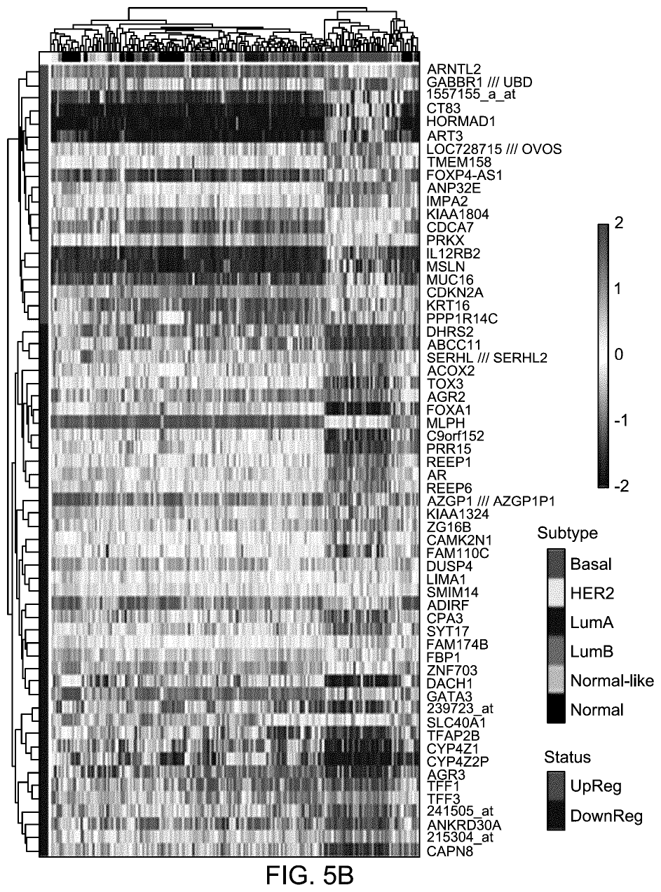

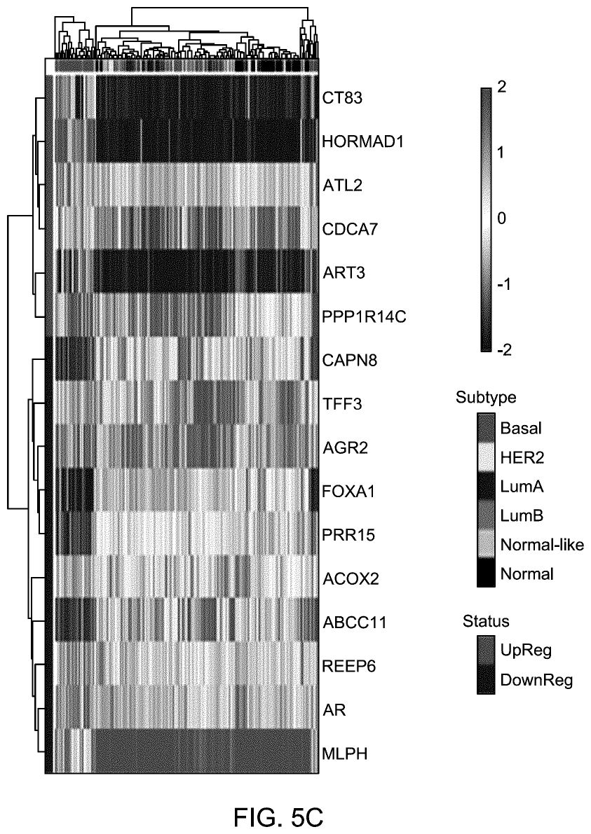

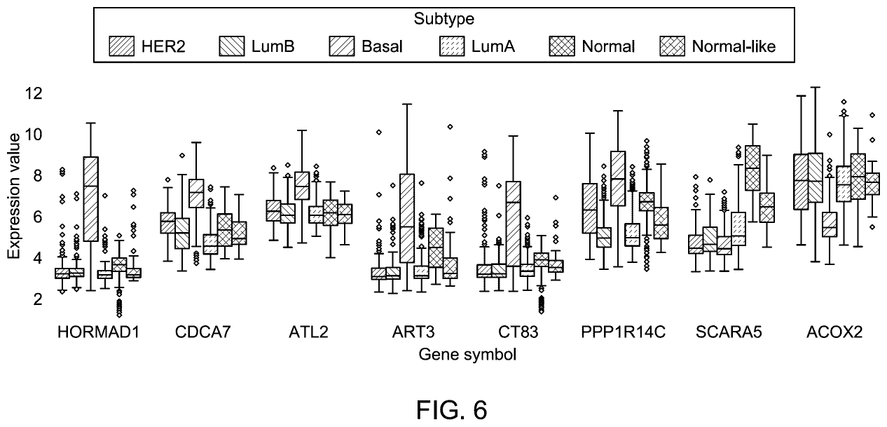

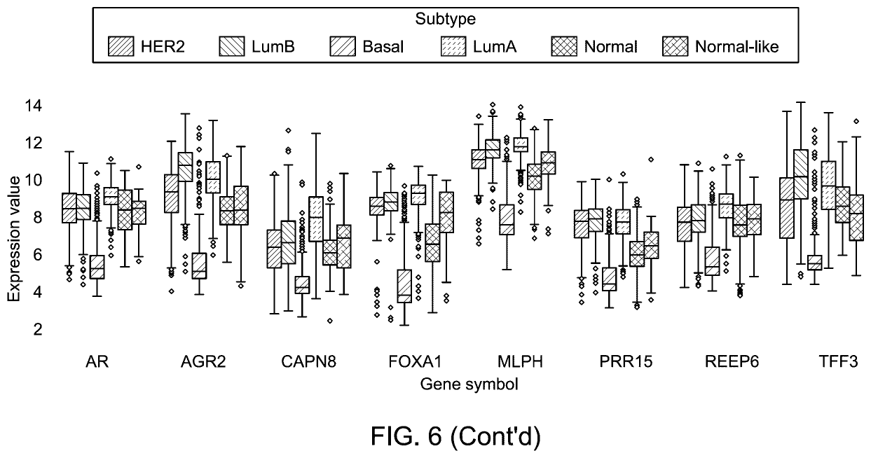

A basal-like diagnostic model including a 15-gene signature (CDCA7, HORMAD1, AR, MLPH, TFF3, AGR2, ATL2, ACOX2, REEP6, CAPN8, FOXA1, CT83, ART3, PPP1R14C, PRR15) was developed, capable of differentiating basal-like subtypes from other breast cancer subtypes as well as normal tissues. This differentiation was achieved using a voting score from five machine learning methods (Support Vector Machine, L1 Regularized SVM, Random Forest, Extremely Randomized Trees, and XGBoost) alongside the expression pattern of 61 differentially expressed genes specific to the basal-like subtype across all samples (Table 3; FIGS. 5A-5C, FIG. 6). HORMAD1, AR, MLPH, AGR2, CT83, and CDCA7 exhibited high importance as predicted by all five machine learning methods. In contrast, TFF3, ATL2, ACOX2, CAPN8, FOXA1, and PPP1R14C genes were predicted by at least two machine-learning methods.

TABLE 3
Voting scores of machine learning methods (Support Vector Machine, L1 Regularized
SVM, Random Forest, Extremely Randomized Trees, XGBoost) and expression values
(fold change and p-value) for basal-like diagnostic gene-signature

| | | | | | Extra | | Votes of ML |
|---|---|---|---|---|---|---|---|
| Gene Symbol | Description | SVM | L1_SVM | RF | trees | XGBoost | methods |
| HORMAD1 | HORMA domain containing 1 | 1 | 1 | 1 | 1 | 1 | 5 |
| AR | androgen receptor | 1 | 1 | 1 | 1 | 1 | 5 |
| MLPH | melanophilin | 1 | 1 | 1 | 1 | 1 | 5 |
| AGR2 | anterior gradient 2 | 1 | 1 | 1 | 1 | 1 | 5 |
| CT83 | cancer/testis antigen 83 | 1 | 1 | 1 | 1 | 1 | 5 |
| CDCA7 | cell division cycle associated 7 | 1 | 1 | 1 | 1 | 1 | 5 |
| TFF3 | trefoil factor 3 (intestinal) | 1 | 0 | 1 | 1 | 1 | 4 |
| FOXA1 | forkhead box A1 | 1 | 0 | 1 | 1 | 1 | 4 |
| ART3 | ADP-ribosyltransferase 3 | 1 | 0 | 1 | 1 | 1 | 4 |
| PRR15 | proline rich 15 | 1 | 0 | 1 | 1 | 1 | 4 |
| ATL2 | atlastin GTPase 2 | 1 | 0 | 1 | 1 | 1 | 4 |
| ACOX2 | acyl-CoA oxidase 2, | 1 | 1 | 1 | 0 | 1 | 4 |
| | branched chain | | | | | | |
| REEP6 | receptor accessory protein 6 | 1 | 0 | 1 | 0 | 0 | 2 |
| CAPN8 | calpain 8 | 0 | 0 | 1 | 1 | 0 | 2 |
| PPP1R14C | protein phosphatase 1, | 0 | 0 | 1 | 1 | 1 | 3 |
| | regulatory (inhibitor) | | | | | | |
| | subunit 14C | | | | | | |

### Example 24: Survival Analysis to Basal-Like Associated Genes with Prognostic Importance

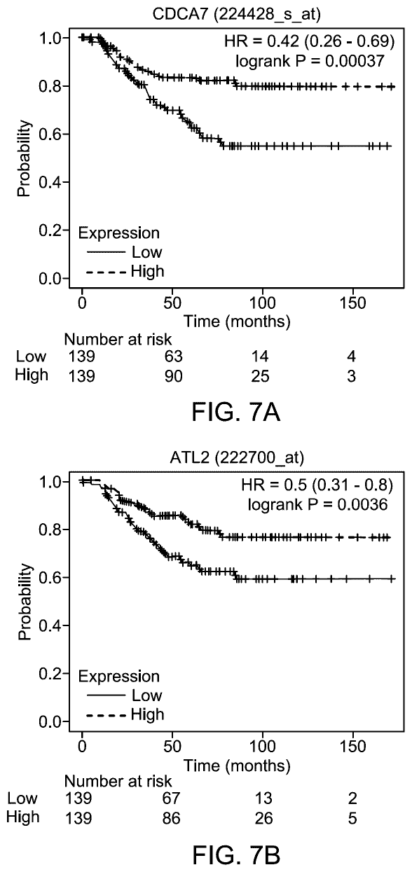

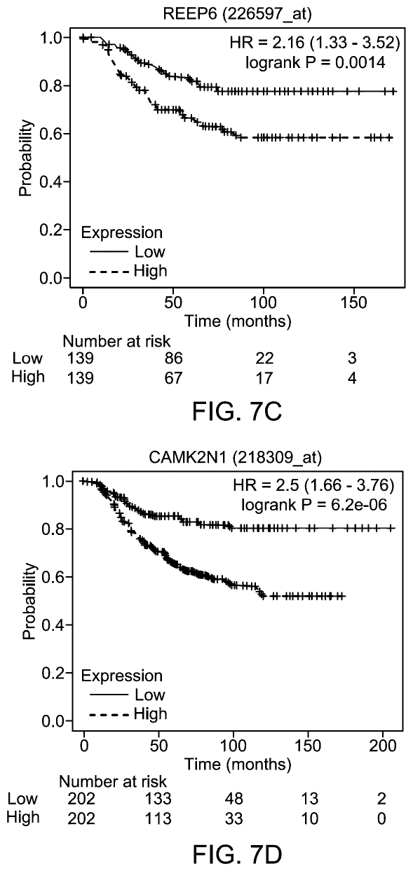

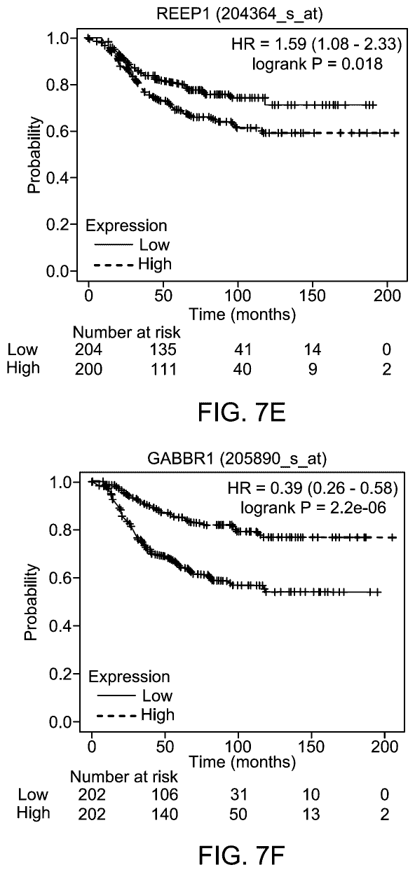

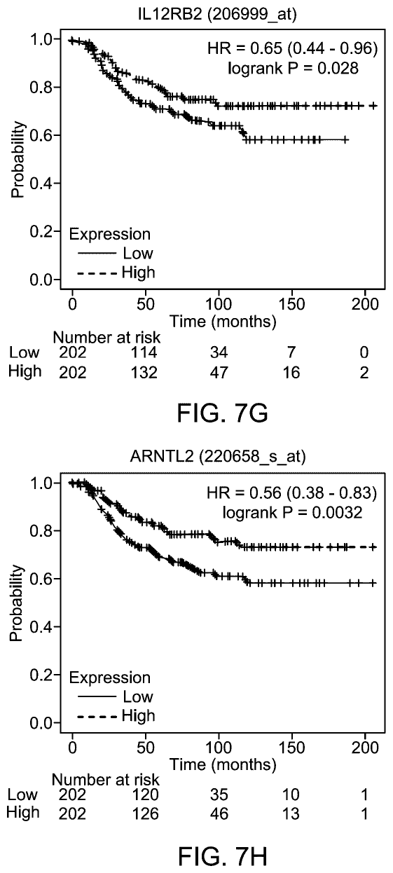

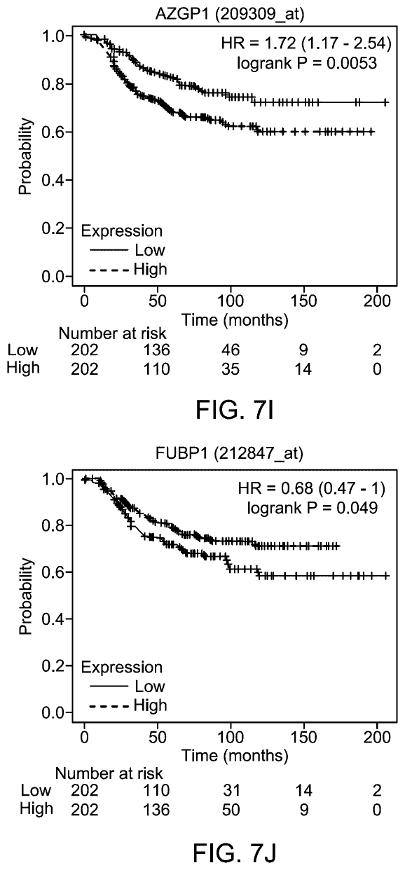

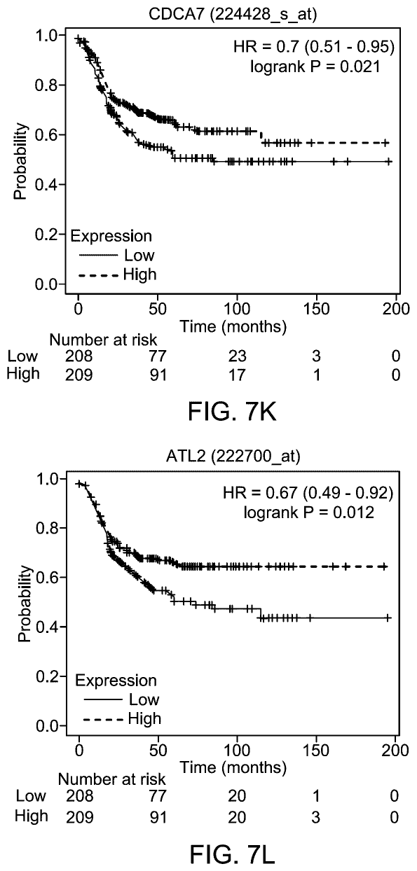

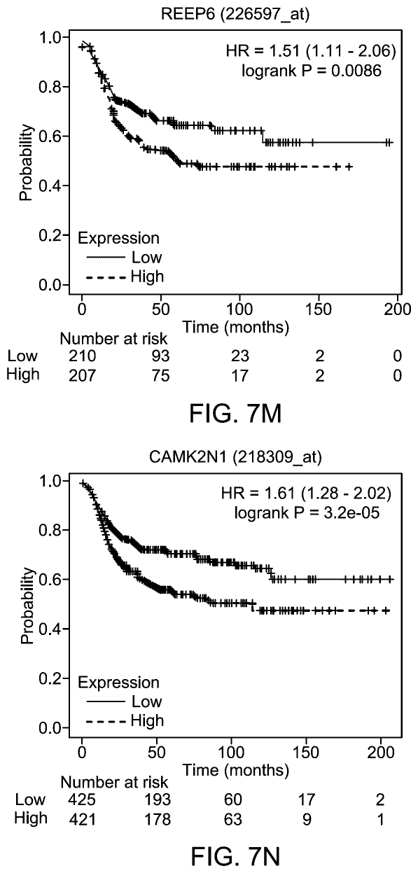

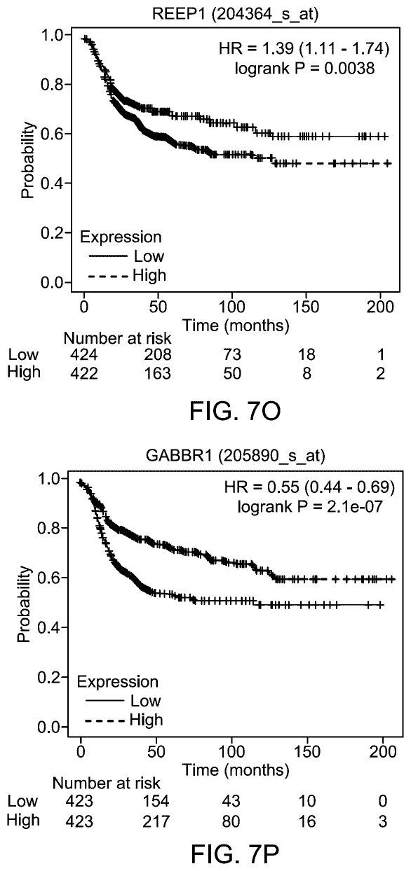

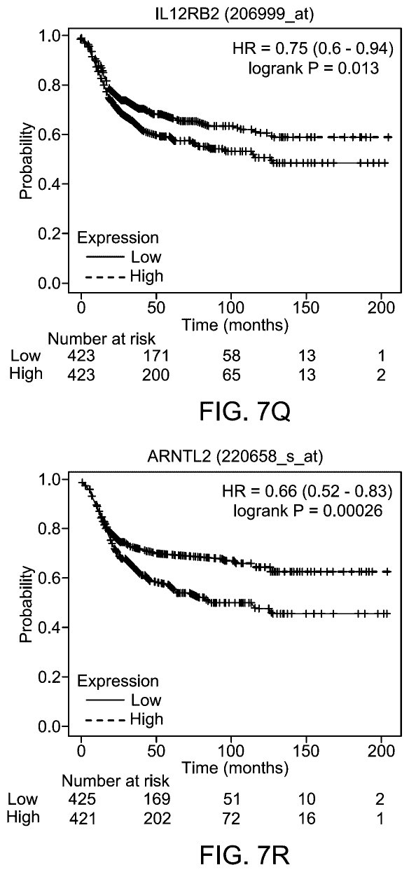

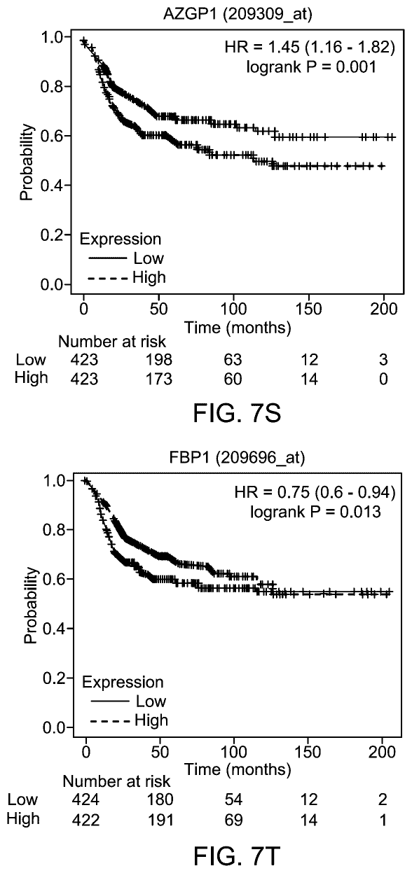

Survival analysis using the Kaplan-Meier estimator was conducted for 61 basal-like associated genes. Statistically significant results were identified for fourteen genes (CDCA7, ATL2, REEP6, CAMK2N1, REEP1, KRT16, GABBR1, IL12RB2, ARNTL2, AZGP1, FBP1, SHISA2, FAM110C, and DACH1) in relation to relapse-free survival during postoperative follow-up, and for seventeen genes (CDCA7, MLPH, ATL2, REEP6, LIMA1, CAMK2N1, REEP1, GABBR1, TMEM158, IL12RB2, ARNTL2, FAM174B, AZGP1, FUBP1, TFAP2B, DHRS2, and CYP4Z1) concerning overall survival under the ‘mRNA (gene chip) subtype—StGallen: Basal’ category by Kaplan-Meier analysis. The hazard ratio (HR) was utilized to compare the risk of death during postoperative follow-up among differentially expressed genes, with values greater or less than one indicating an association between altered gene expression and poor survival. Narrow confidence intervals (95% CI), along with a large sample size, suggest precise and reliable estimates of survival probability. The final statistical significance of patient survival for high and low-expression groups was measured using the log-rank p-value. Ten genes (CDCA7, ATL2, REEP6, CAMK2N1, REEP1, GABBR1, IL12RB2, ARNTL2, AZGP1, and FBP1) were found to show significance in both relapse-free survival and overall survival, indicating their potential as novel prognostic gene signatures for the basal-like subtype in breast cancer (Table 4, FIGS. 7A-7T).

TABLE 4
List of 21 prognostic significant genes for basal-like subtypes with Hazard Ratio,
95% Confidence Interval, and Log-rank p-value: Top ten genes showing prognostic
significance in both relapse-free survival (RFS) and overall survival (OS) analysis,
while additional four and seven genes were in RFS and OS respectively.

| | Gene | Hazard Ratio | | 95% Confidence Interval | | Log rank p value | |
|---|---|---|---|---|---|---|---|
| Probe ID | Symbol | RFS | OS | RFS | OS | RFS | OS |

Ten genes significant in RFS and OS

| | | | | | | | |
|---|---|---|---|---|---|---|---|
| 224428_s_at | CDCA7 | 0.7 | 0.42 | (0.51-0.95) | (0.26-0.69) | 0.0208 | 0.0004 |
| 222700_at | ATL2 | 0.67 | 0.5 | (0.49-0.92) | (0.31-0.8) | 0.0124 | 0.0036 |
| 226597_at | REEP6 | 1.51 | 2.16 | (1.11-2.06) | (1.33-3.52) | 0.0086 | 0.0014 |
| 218309_at | CAMK2N1 | 1.61 | 2.5 | (1.28-2.02) | (1.66-3.76) | 0.00003 | 0.0000062 |
| 204364_s_at | REEP1 | 1.39 | 1.59 | (1.11-1.74) | (1.08-2.33) | 0.0038 | 0.0181 |
| 205890_s_at | GABBR1 | 0.55 | 0.39 | (0.44-0.69) | (0.26-0.58) | 0.0000002 | 0.000002 |
| 206999_at | IL12RB2 | 0.75 | 0.65 | (0.6-0.94) | (0.44-0.96) | 0.013 | 0.0282 |
| 220658_s_at | ARNTL2 | 0.66 | 0.56 | (0.52-0.83) | (0.38-0.83) | 0.00026 | 0.0032 |
| 209309_at | AZGP1 | 1.45 | 1.72 | (1.16-1.82) | (1.17-2.54) | 0.001 | 0.0053 |
| 209696_at | FBP1 | 0.75 | 0.68 | (0.6-0.94) | (0.47-1) | 0.0125 | 0.0493 |

Four genes significant in RFS only

| | | | | |
|---|---|---|---|---|
| 230493_at | SHISA2 | 1.71 | (0.52-0.97) | 0.0293 |
| 226863_at | FAM110C | 1.6 | (1.17-2.18) | 0.0029 |
| 205472_s_at | DACH1 | 1.43 | (1.14-1.79) | 0.0016 |
| 209800_at | KRT16 | 1.33 | (1.06-1.66) | 0.013 |

Seven genes significant in OS only

| | | | | |
|---|---|---|---|---|
| 218211_s_at | MLPH | 1.52 | (1.03-2.24) | 0.0318 |
| 222456_s_at | LIMA1 | 1.65 | (1.02-2.66) | 0.0377 |
| 213338_at | TMEM158 | 0.66 | (0.45-0.98) | 0.036 |
| 221880_s_at | FAM174B | 1.62 | (1.1-2.38) | 0.0137 |
| 1553394_a_at | TFAP2B | 1.91 | (1.18-3.1) | 0.0071 |
| 206463_s_at | DHRS2 | 1.67 | (1.13-2.46) | 0.0096 |
| 237395_at | CYP4Z1 | 1.77 | (1.1-2.85) | 0.0179 |

### Example 25: Drug Target Identification

Fifty two UniProt IDs were identified among 61 hub genes resulting in the identification of six drug targets, including AR, FBP1, IMPA2, KRT16, TFF1, and MSLN, in DrugBank (Table 5).

TABLE 5
Machine learning algorithms for validation

| Symbol | UniProt Name | UniProt ID | DrugBank ID: Drug Name |
|---|---|---|---|
| MSLN | Mesothelin | Q13421 | DB12845: Amatuximab |
| TFF1 | Trefoil factor 1 | P04155 | DB00481: Raloxifene, DB04468: Afimoxifene |
| KRT16 | Keratin, type I | P08779 | DB01593: Zinc, DB14487: Zinc acetate, |
| | cytoskeletal 16 | | DB14533: Zinc chloride |
| IMPA2 | Inositol | O14732 | DB01356: Lithium cation, DB14507: |
| | monophosphatase 2 | | Lithium citrate, DB14508: Lithium |
| | | | succinate, DB14509: Lithium carbonate |
| FBP1 | Fructose-1,6- | P09467 | DB00131: Adenosine phosphate, DB02778: 2,5-Anhydroglucitol- |
| | bisphosphatase 1 | | 1,6-Biphosphate), DB02848 ({4-[3-(6,7-Diethoxy-Quinazolin-4- |
| | | | Ylamino)-Phenyl]-Thiazol-2-Yl}-Methanol, DB04175: Mdl-29951, |
| | | | DB04493: Fructose-6-Phosphate, DB05053: MB-07803, DB05518: |
| | | | Managlinat dialanetil, DB07270: N-[7-(3-AMINOPHENYL)-5- |
| | | | METHOXY-1,3-BENZOXAZOL-2-YL]-2,5- |
| | | | DICHLOROBENZENESULFONAMIDE, DB07312: 2,5- |
| | | | DICHLORO-N-(5-CHLORO-1,3-BENZOXAZOL-2- |
| | | | YL)BENZENESULFONAMIDE, DB07321: 2,5-DICHLORO-N- |
| | | | [5-METHOXY-7-(6-METHOXYPYRIDIN-3-YL)-1,3- |
| | | | BENZOXAZOL-2-YL]BENZENESULFONAMIDE, DB08484: |
| | | | 4-AMINO-N-[(2- |
| | | | SULFANYLETHYL)CARBAMOYL]BENZENESULFONAMIDE |
| AR | Androgen | P10275 | DB00255: Diethylstilbestrol), DB00367: Levonorgestrel, |
| | receptor | | DB00396: Progesterone, DB00421: Spironolactone, DB00499: |
| | | | Flutamide, DB00621: Oxandrolone, DB00623: Fluphenazine, |
| | | | DB00624: Testosterone, DB00648: Mitotane, DB00655: Estrone, |
| | | | DB00665: Nilutamide, DB00675: Tamoxifen; DB00687: |
| | | | Fludrocortisone, DB00858: Drostanolone, DB00957: Norgestimate, |
| | | | DB00984: Nandrolone phenpropionate, DB01026: Ketoconazole: |
| | | | DB01063: Acetophenazine, DB01128: Bicalutamide, DB01185: |
| | | | Fluoxymesterone, DB01395: Drospirenone, DB01406: Danazol, |
| | | | DB01420: Testosterone propionate, DB01428: Oxybenzone, |
| | | | DB01481: 1-Testosterone, DB01541: Boldenone, DB01564: |
| | | | Calusterone, DB01608: Periciazine, DB01708: Prasterone, |
| | | | DB02266: Flufenamic Acid, DB02901: Stanolone, DB02932: (R)- |
| | | | Bicalutamide, DB02998: Methyltrienolone etc etc. |

Machine learning algorithms for validation involved the assessment of a basal-like diagnostic model including a 15-gene signature through both internal and external validation approaches utilizing a new set of eight ML algorithms (Extra Trees, Gradient Boosting, kNN, Logistic Regression, Neural Network, Random Forest, SVM, and XGB Classifier). Initially, a 10-fold cross-validation technique was employed to evaluate the performance of ML models for gene signatures using a mean of 1,279 BC samples. The dataset was divided into 10 equal parts (folds), using nine parts for training and one for validation. This process was repeated 10 times for each fold, and the results were averaged to obtain a more reliable estimate of model performance.

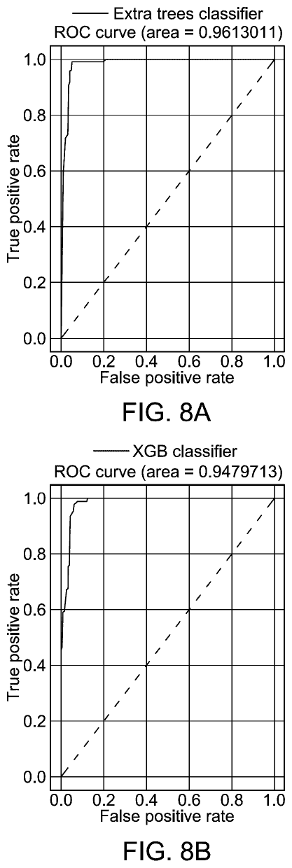

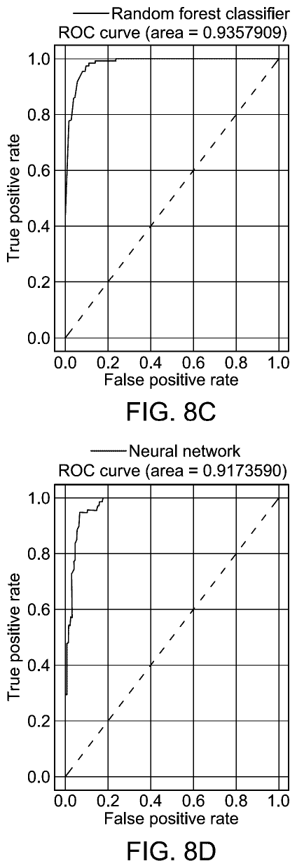

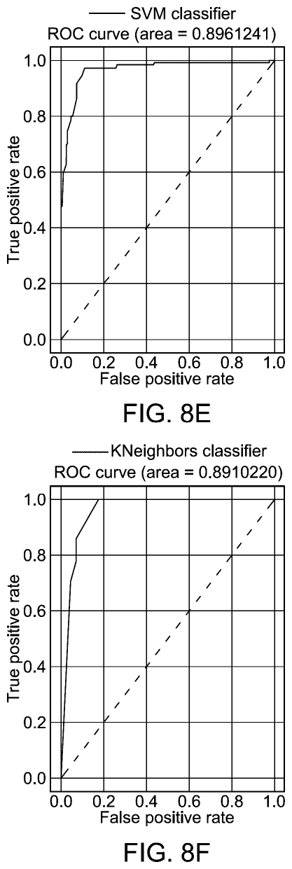

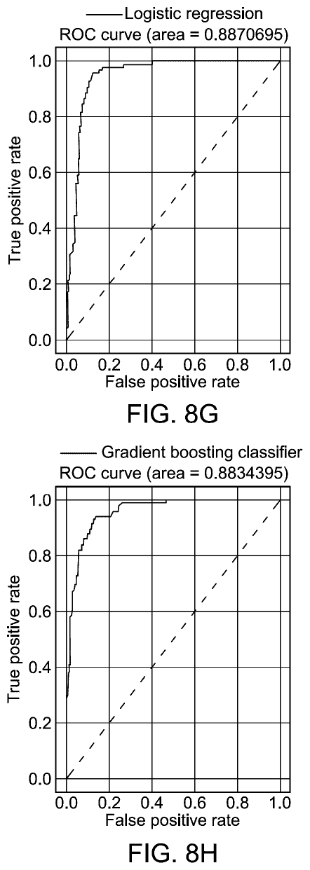

Subsequently, external validation was performed on two independent datasets including 351 BC samples to validate the diagnostic performance of the fifteen-gene signature using the same set of eight ML algorithms. Each ML model's performance was evaluated by measuring various performance metrics, including accuracy, precision, recall, F1 score, and ROC_AUC scores. For internal validation, ROC_AUC scores were 0.98 for Extra Trees, XGB, Random Forest, SVM, and Gradient Boosting Classifier. In external validation, the Extra Trees Classifier achieved the highest ROC_AUC score of 0.96, followed by the XGB Classifier (0.95) and the Random Forest Classifier (0.94) (Table 6, FIGS. 8A-8H). The ML model with the highest ROC_AUC indicated the candidate gene signatures as potential biomarkers. The F1 score was highest in Random Forest for both internal (0.95) and external validation (0.92), indicating it as the best model among the nine ML classifiers.

TABLE 6
Validation of machine learning model by measuring accuracy, precision, recall, F1 and
ROC_AUC scores: internal validation with 10-fold cross validation means of 1279 breast
cancer samples and external validation with two independent datasets of 351 breast cancer samples.

| | Preformation matrix and validation types | | | | | | | | | |
|---|---|---|---|---|---|---|---|---|---|---|
| | Accuracy | | Precision | | Recall | | F1 | | ROC_AUC | |
| ML Algorithms | Int* | Ext^{#} | Int | Ext | Int | Ext | Int | Ext | Int | Ext |
| Extra Trees Classifier | 0.96 | 0.95 | 0.92 | 0.85 | 0.89 | 0.99 | 0.94 | 0.91 | 0.98 | 0.96 |
| XGB Classifier | 0.97 | 0.94 | 0.90 | 0.85 | 0.89 | 0.96 | 0.95 | 0.90 | 0.98 | 0.95 |
| Random Forest Classifier | 0.97 | 0.93 | 0.89 | 0.84 | 0.89 | 0.94 | 0.96 | 0.92 | 0.98 | 0.94 |
| Neural Network | 0.97 | 0.93 | 0.87 | 0.85 | 0.87 | 0.90 | 0.95 | 0.91 | 0.97 | 0.92 |
| SVM Classifier | 0.96 | 0.91 | 0.84 | 0.82 | 0.87 | 0.87 | 0.95 | 0.91 | 0.98 | 0.90 |
| K-Nearest Neighbor | 0.96 | 0.91 | 0.84 | 0.82 | 0.85 | 0.86 | 0.94 | 0.89 | 0.96 | 0.89 |
| Logistic Regression | 0.96 | 0.90 | 0.83 | 0.80 | 0.86 | 0.86 | 0.94 | 0.90 | 0.97 | 0.89 |
| Gradient Boosting Classifier | 0.96 | 0.89 | 0.81 | 0.75 | 0.89 | 0.88 | 0.94 | 0.90 | 0.98 | 0.88 |

*Internal validation,
^{#}External validation

### Example 26: Validation by RNA-Seq and Protein Expression Data

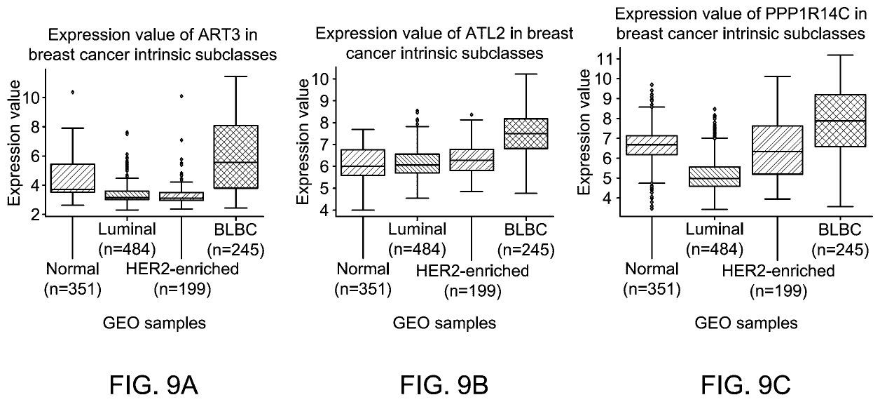

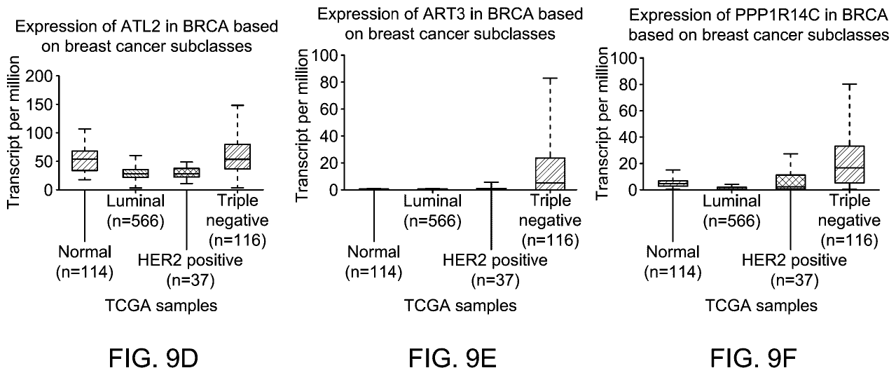

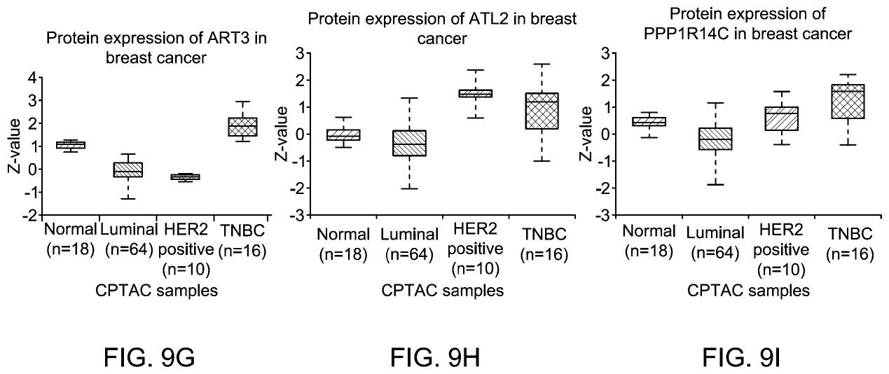

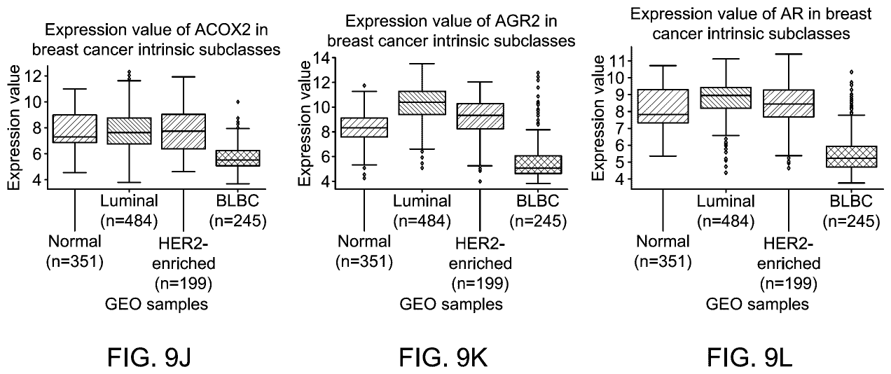

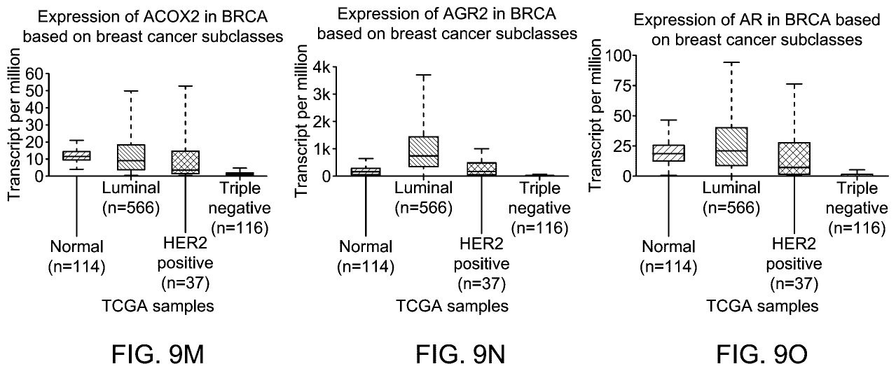

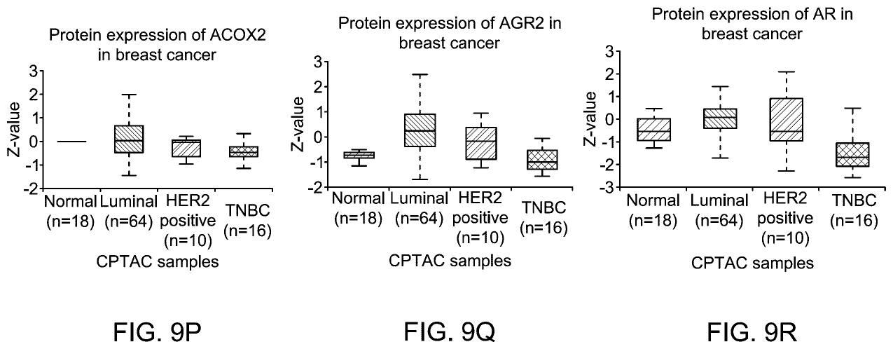

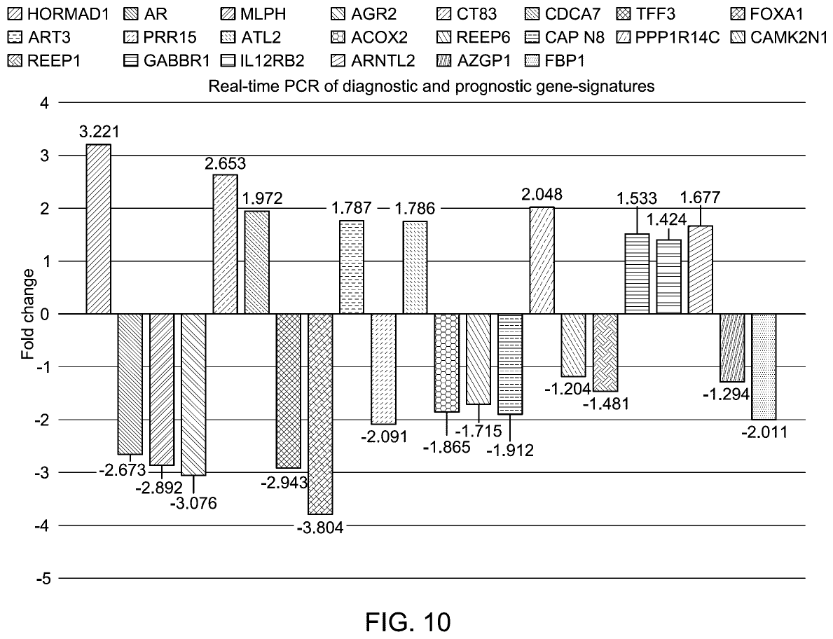

UALCAN portal was used to confirm the microarray-based gene expression pattern of the basal-like gene signature using TCGA (RNA expression data by RNA-seq) and CPTAC (protein expression data by mass spectrometry) databases. BC intrinsic subtypes were arranged into four major classes: normal (normal and normal-like), luminal (lumA and lumB), HER2-enriched, and basal-like BC (BLBC). Despite reported differences in molecular-based intrinsic and IHC-based pathological subtyping, BLBC are considered equivalent to triple-negative BC (TNBC) in broader contexts. RNA-seq data confirmed the expression pattern of all 22 identified genes, showing upregulation of HORMAD1, CT83, PPP1R14C, CDCA7, ART3, ATL2, ARNTL2, GABBR1, and IL12RB2, alongside downregulation of FOXA1, AGR2, TFF3, MLPH, AR, PRR15, FBP1, CAPN8, ACOX2, REEP6, REEP1, AZGP1, and CAMK2N1. However, mass spectrometry-based protein expression data confirmed only sixteen genes, including three upregulated (PPP1R14C, ART3, and ATL2) and thirteen downregulated (FOXA1, AGR2, TFF3, MLPH, AR, PRR15, FBP1, CAPN8, ACOX2, REEP6, REEP1, AZGP1, and CAMK2N1), as protein expression data was not available for the remaining six genes (HORMAD1, CT83, CDCA7, ARNTL2, GABBR1, and IL12RB2) (FIGS. 9A-9R).

Validation by Real Time qPCR analysis involved using real-time qPCR to confirm the identified basal-like subtype biomarkers (gene signatures) by determining the relative expression of 22 genes (fifteen-gene signature for diagnosis: CDCA7, HORMAD1, AR, MLPH, TFF3, AGR2, ATL2, ACOX2, REEP6, CAPN8, FOXA1, CT83, ART3, PPP1R14C, PRR15; and ten-gene signature for prognosis: CDCA7, ATL2, REEP6, CAMK2N1, REEP1, GABBR1, IL12RB2, ARNTL2, AZGP1, FBP1) with overlap among CDCA7, ATL2, and REEP6 (FIG. 10). GAPDH was used as an internal control. qPCR results were in concordance with microarray-analyzed expression patterns, demonstrating upregulation of HORMAD1, CT83, PPP1R14C, CDCA7, ART3, ATL2, ARNTL2, GABBR1, and IL12RB2, and downregulation of ACOX2, AGR2, AR, FOXA1, TFF3, MLPH, PRR15, FBP1, CAPN8, REEP6, REEP1, AZGP1, and CAMK2N1 (Table 7).

TABLE 7
Expression of diagnostic and prognostic gene signatures
in microarray and RT-qPCR: Results of Microarray
and RT-qPCR were represented by fold change (FC),
quantitative expression (Rq) and P-values.

| | Microarray | | RT-qPCR | | |
|---|---|---|---|---|---|
| Genes | FC | Adj. P. Val | Rq | FC | P. Va |

Expression of Diagnostic gene Signature

| | | | | | |
|---|---|---|---|---|---|
| HORMAD1 | 3.538 | 7.80E−262 | 9.325 | 3.221 | 0.01721 |
| AR | −2.922 | 4.79E−220 | 0.156 | −2.673 | 5.44E−05 |
| MLPH | −3.163 | 1.06E−231 | 0.134 | −2.892 | 7.30E−17 |
| AGR2 | −3.737 | 7.69E−193 | 0.118 | −3.076 | 0.00769 |
| CT83 | 2.481 | 2.35E−168 | 6.292 | 2.653 | 0.00059 |
| CDCA7 | 1.858 | 3.52E−157 | 3.923 | 1.972 | 1.37E−07 |
| TFF3 | −3.209 | 8.52E−131 | 0.129 | −2.943 | 0.00038 |
| FOXA1 | −3.564 | 1.56E−186 | 0.0715 | −3.804 | 7.55E−08 |
| ART3 | 2.31 | 2.60E−115 | 3.451 | 1.787 | 2.97E−05 |
| PRR15 | −2.374 | 3.35E−139 | 0.234 | −2.091 | 5.62E−05 |
| ATL2 | 1.289 | 3.58E−116 | 3.45 | 1.786 | 3.15E−06 |
| ACOX2 | −2.027 | 2.28E−98 | 0.274 | −1.865 | 0.00017 |
| REEP6 | −2.189 | 2.02E−120 | 0.3045 | −1.715 | 3.94E−05 |
| CAPN8 | −2.314 | 6.76E−97 | 0.2656 | −1.912 | 1.42E−05 |
| PPP1R14C | 1.882 | 5.79E−82 | 4.137 | 2.048 | 0.00547 |

Expression of Prognostic Gene Signature

| | | | | | |
|---|---|---|---|---|---|
| CDCA7 | 1.858 | 3.52E−157 | 3.923 | 1.972 | 1.37E−07 |
| ATL2 | 1.289 | 3.58E−116 | 3.45 | 1.786 | 3.15E−06 |
| REEP6 | −2.189 | 2.02E−120 | 0.304 | −1.715 | 3.94E−05 |
| CAMK2N1 | −1.412 | 2.42E−104 | 0.434 | −1.204 | 1.89E−06 |
| REEP1 | −1.786 | 2.16E−86 | 0.3582 | −1.481 | 0.0001 |
| GABBR1 | 1.731 | 4.92E−45 | 2.895 | 1.533 | 0.0279 |
| IL12RB2 | 1.362 | 1.66E−91 | 2.6841 | 1.424 | 0.0396 |
| ARNTL2 | 1.205 | 4.31E−79 | 3.198 | 1.677 | 0.0138 |
| AZGP1 | −1.727 | 1.16E−46 | 0.407 | −1.294 | 0.0031 |
| FBP1 | −1.73 | 1.10E−91 | 0.2479 | −2.011 | 0.0002 |

In the present disclosure, the integration of artificial intelligence, ML algorithms, bioinformatics, and gene expression profiling data could lead to the identification of diagnostic and prognostic biomarkers for basal-like, the most aggressive BC subtypes. Hierarchical clustering and PCA plot demonstrated the capability of a 15-gene signature to accurately and distinctly differentiate basal-like from other BC subtypes as well as healthy controls, thereby improving diagnosis. KM plotter analysis for basal-like BC has revealed 10 genes predicting the worst RFS and OS. High-performance matrix scores of ML algorithms indicate immense potential for the diagnostic and prognostic gene signature to be used in pathology laboratories and clinically applied to predict prognostic outcomes for BLBC. Quantitative PCR has validated these findings; however, subsequent validation of FFPE tissue on large cohorts of patients from different populations will expand acceptability and could assist in individualizing treatment decisions for women with BLBC. Ultimately, the findings presented in this disclosure underscore the transformative potential of advanced computational methods in oncology, paving the way for more personalized and effective treatment strategies that can significantly improve patient outcomes in the face of aggressive breast cancer subtypes.

Numerous modifications and variations of the present disclosure are possible in light of the above teachings. It is therefore to be understood that within the scope of the appended claims, the invention may be practiced otherwise than as specifically described herein.

1. A method for treating a breast cancer in a subject in need thereof, the method comprising:
assaying a breast tumor sample from the subject for gene expression levels of
a diagnostic gene set comprising at least two genes selected from the group consisting of CDCA7, HORMAD1, AR, MLPH, TFF3, AGR2, ATL2, ACOX2, REEP6, CAPN8, FOXA1, CT83, ART3, PPP1R14C, and PRR15, and
a prognostic gene set comprising at least two genes selected from the group consisting of CDCA7, ATL2, REEP6, CAMK2N1, REEP1, GABBR1, IL12RB2, ARNTL2, AZGP1, and FBP1,
selecting a treatment option which is least one selected from the group comprising surgery and an anticancer agent based on the gene expression levels, and
treating the patient with the treatment option.

2. The method of claim 1, wherein
the breast cancer is determined to be a basal-like breast cancer based on the gene expression levels of the diagnostic gene set.

3. The method of claim 1, wherein
the anticancer agent is at least one selected from the group consisting of an anticancer agent targeting MSLN, an anticancer agent targeting TFF1, an anticancer agent targeting KRT16, an anticancer agent targeting IMPA2, an anticancer agent targeting FBP1, an anticancer agent targeting AR.

4. The method of claim 1, wherein
the anticancer agent is selected based on the gene expression levels of the prognostic gene set.

5. The method of claim 3, wherein
the anticancer agent targeting MSLN is amatuximab.

6. The method of claim 3, wherein
the anticancer agent targeting TFF1 is at least one selected from the group consisting of raloxifene and afimoxifene.

7. The method of claim 3, wherein
the anticancer agent targeting KRT16 is at least one selected from the group consisting of zinc, zinc acetate, and zinc chloride.

8. The method of claim 3, wherein
the anticancer agent targeting IMPA2 is at least one selected from the group consisting of lithium cation, lithium citrate, lithium succinate, and lithium carbonate.

9. The method of claim 3, wherein
the anticancer agent targeting FBPI is at least one selected from the group consisting of adenosine phosphate, Mdl-29951, fructose-6-phosphate, MB-07803, and managlinat dialanetil.

10. The method of claim 3, wherein
the anticancer agent targeting AR is at least one selected from the group consisting of diethylstilbestrol, levonorgestrel, progesterone, spironolactone, flutamide, oxandrolone, and fluphenazine.

11. The method of claim 1, wherein
the diagnostic gene set comprises at least five genes selected from the group consisting of CDCA7, HORMAD1, AR, MLPH, TFF3, AGR2, ATL2, ACOX2, REEP6, CAPN8, FOXA1, CT83, ART3, PPP1R14C, and PRR15.

12. The method of claim 1, wherein
the diagnostic gene set comprises at least seven genes selected from the group consisting of CDCA7, HORMAD1, AR, MLPH, TFF3, AGR2, ATL2, ACOX2, REEP6, CAPN8, FOXA1, CT83, ART3, PPP1R14C, and PRR15.

13. The method of claim 1, wherein
the diagnostic gene set comprises at least nine genes selected from the group consisting of CDCA7, HORMAD1, AR, MLPH, TFF3, AGR2, ATL2, ACOX2, REEP6, CAPN8, FOXA1, CT83, ART3, PPP1R14C, and PRR15.

14. The method of claim 1, wherein
the diagnostic gene set comprises at least eleven genes selected from the group consisting of CDCA7, HORMAD1, AR, MLPH, TFF3, AGR2, ATL2, ACOX2, REEP6, CAPN8, FOXA1, CT83, ART3, PPP1R14C, and PRR15.

15. The method of claim 1, wherein
the diagnostic gene set comprises CDCA7, HORMAD1, AR, MLPH, TFF3, AGR2, ATL2, ACOX2, REEP6, CAPN8, FOXA1, CT83, ART3, PPP1R14C, and PRR15.

16. The method of claim 1, wherein
the prognostic gene set comprises at least three genes selected from the group consisting of CDCA7, ATL2, REEP6, CAMK2N1, REEP1, GABBR1, IL12RB2, ARNTL2, AZGP1, and FBP1.

17. The method of claim 1, wherein
the prognostic gene set comprises at least five genes selected from the group consisting of CDCA7, ATL2, REEP6, CAMK2N1, REEP1, GABBR1, IL12RB2, ARNTL2, AZGP1, and FBP1.

18. The method of claim 1, wherein
the prognostic gene set comprises at least seven genes selected from the group consisting of CDCA7, ATL2, REEP6, CAMK2N1, REEP1, GABBR1, IL12RB2, ARNTL2, AZGP1, and FBP1.

19. The method of claim 1, wherein
the prognostic gene set comprises at least nine genes selected from the group consisting of CDCA7, ATL2, REEP6, CAMK2N1, REEP1, GABBR1, IL12RB2, ARNTL2, AZGP1, and FBP1.

20. The method of claim 1, wherein
the prognostic gene set comprises CDCA7, ATL2, REEP6, CAMK2N1, REEP1, GABBR1, IL12RB2, ARNTL2, AZGP1, and FBP1.
